(19) United States
(12) Patent Application Publication
(10) Pub. No.: US 2026/0226402 A1
(43) Pub. Date: Aug. 6, 2026

(54) IMPROVED MICROBIAL GROWTH MEDIA

(71) Applicant: Becton, Dickinson and Company, Franklin Lakes, NJ (US)

(72) Inventors: Gretta L. Koontz, Glen Rock, PA (US); Stephanie Ann Frey, York, PA (US); Kristina Anne Robinson, Parkville, MD (US)

(21) Appl. No.: 19/152,880

(22) PCT Filed: Jan. 30, 2024
(86) PCT No.: PCT/US2024/013547

§ 371 Date: Jul. 31, 2025

Related U.S. Application Data
(60) Provisional application No. 63/482,760, filed on Feb. 1, 2023.

(51) Int. Cl. C12N 1/16; C12M 1/36; C12N 1/20

(52) U.S. Cl. CPC C12N 1/16; C12M 41/48; C12N 1/20

(57) ABSTRACT

The compositions, apparatuses, and methods described herein relate to the discovery that the time to detection of yeast and microorganism contaminants in microbial growth media can be improved by controlling the concentration of iron and copper.

### CROSS-REFERENCE TO RELATED APPLICATIONS

This application claims priority to the U.S. Provisional Patent Application Ser. No. 63/482,760, filed on Feb. 1, 2023, which is hereby incorporated by reference herein in its entirety.

### FIELD

Embodiments described herein generally relate to an enhanced microbial growth media providing an increased growth rate and/or reduced time to detection of yeast and/or other microorganisms.

### BACKGROUND

Current microbial growth media contains yeast nitrogen base without amino acids, which is comprised of unspecified amounts of iron, copper, and other nutrients. At the volumes required for full batch production, the presence of yeast nitrogen base results in precipitation of iron in the media. The undefined concentrations of iron and copper in existing microbial growth media delays the time to detection of certain microbes. Thus, there is a need in the field for an improved microbial growth media.

### SUMMARY

The compositions, apparatuses, and methods described herein relate to the discovery that the growth rate and/or time to detection of yeast and/or other microorganism in microbial growth media can be improved by controlling the concentration of iron and optionally copper.

Embodiments provided herein include the following numbered Embodiments:
1. A microbial growth media providing an increased growth rate of Candida, the microbial growth media comprising:
   a concentration of iron of about 1 to about 400 μM; and
   optionally, a concentration of copper of up to about 700 μM;
   wherein the growth rate of Candida cultured in the microbial growth media is increased as compared to the same microbial growth media in the absence of the iron and copper.
2. The microbial growth media of embodiment 1, wherein the time to detection (TTD) of Candida cultured in the microbial growth media is reduced as compared to the same microbial growth media in the absence of the iron and copper.
3. The microbial growth media of any one of the preceding embodiments, wherein the reduced TTD of Candida cultured in the microbial growth media is determined by:
   amending culture containers comprising the microbial growth media with and without the iron and copper with 0.5, 3, or 10 ml of blood;
   inoculating the culture containers with 0.1 mL of a culture of Candida to provide 10-100 CFU of Candida per container;
   culturing the Candida at about 30° C. to 37° C. for up to 120 hours;
   monitoring the culture containers for a signal indicative of the presence of Candida; and
   determining the TTD of the Candida in the microbial growth media with and without the iron and copper.
4. The microbial growth media of any one of the preceding embodiments, wherein the Candida is Candida albicans or Candida glabrata.
5. The microbial growth media of any one of the preceding embodiments, wherein the microbial growth media comprises, or comprises about, 0, 0.01, 0.1, 0.2, 0.3, 0.4, 0.5, 0.6, 0.7, 0.8, 0.9, or 1.0 g/L yeast nitrogen base, or a range defined by any two of the preceding values.
6. The microbial growth media of any one of the preceding embodiments, wherein the microbial growth media comprises less than 0.01% w/v yeast nitrogen base.
7. The microbial growth media of any one of the preceding embodiments, wherein the yeast nitrogen base does not comprise histidine, methionine, and tryptophan.
8. The microbial growth media of any one of the preceding embodiments, wherein the microbial growth media comprises, or comprises about, 1, 2, 3, 4, 5, 6, 7, 8, 9, 10, 15, 20, 25, 28, 50, 75, 100, 125, 150, 170, 175, 200, 225, 250, 275, 300, 325, 350, or 400 μM iron, or a range defined by any two of the preceding values, optionally 1 to 400 μM, 1 to 350 μM, 1 to 200μM, 1 to 100 μM, 1 to 50 μM, 2 to 400 μM, 2 to 350 μM, 25 to 400 μM, 25 to 325 μM, 25 to 200 μM, 25 to 100 μM, or 25 to 75 μM.
9. The microbial growth media of any one of the preceding embodiments, wherein concentration of iron is about 28 μM.
10. The microbial growth media of any one of the preceding embodiments, wherein concentration of iron is about 100 μM.
11. The microbial growth media of any one of the preceding embodiments, wherein concentration of iron is about 170 μM.
12. The microbial growth media of any one of the preceding embodiments, wherein the iron is ferric iron.
13. The microbial growth media of any one of the preceding embodiments, wherein the iron is provided as ferric ammonium citrate, ferric chloride, ferric sulfate, ferric nitrate.
14. The microbial growth media of any one of the preceding embodiments, wherein the microbial growth media comprises, or comprises about, 0, 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 75, 100, 150, 200, 300, 400, 500, 600, 650, or 700 μM copper, or a range defined by any two of the preceding values, optionally 0 to 700 μM, 0 to 650 μM, 0 to 500 μM, 0 to 300 μM, 0 to 100 μM, 0 to 50 μM, 0 to 25 μM, 0.1 to 100 μM, 0.1 to 50 μM, 0.1 to 25 μM, 0.5 to 650 μM, 0.5 to 300 μM, 0.5 to 100 μM, 0.5 to 50 μM, or 0.5 to 25 μM.
15. The microbial growth media of any one of the preceding embodiments, wherein the concentration of copper is about 3 μM.
16. The microbial growth media of any one of the preceding embodiments, wherein the copper is cupric copper.
17. The microbial growth media of any one of the preceding embodiments, wherein the copper is provided as cupric sulfate, cupric chloride, cupric nitrate, cupric bromide, cupric chlorate, and/or copper(II) gluconate.
18. The microbial growth media of any one of the preceding embodiments, wherein the microbial growth media is an aqueous liquid growth media further comprising sucrose, dextrose, D-Trehalose, yeast extract, L-glutamic acid, tryptic soy broth, sodium polyanethole sulfonate, menadione, pyridoxal HCl, ferulic acid, sodium hydroxide, ascorbic acid, L-Cysteine, hemin.
19. The microbial growth media of any one of the preceding embodiments, wherein the microbial growth medium comprises, or comprises about, 1.0 to 10.0 g/L yeast extract, 0.15 to 1.5 g/L sucrose, 1.0 to 10.0 g/L dextrose, 1.0 to 10.0 g/L D-trehalose, 0.001 to 1.5 g/L L-glutamic acid, 0.00001 to 0.01 g/L L-cysteine, 0.5 to 15 g/L TSB, 0.001 to 1.5 g/L SPS, 0.00001 to 0.01 g/L menadione, 0.0001 to 1 g/L Pyridoxal HCl, 0.0001 to 1 g/L ferulic acid, 0.1 to 10 g/L sodium hydroxide, 0.001 to 1.0 g/L ascorbic acid, and 0.0001 to 1 g/L hemin, in water.
20. The microbial growth media of any one of the preceding embodiments, wherein the microbial growth medium comprises, or comprises about, 0.001 g/L to 0.15 g/L iron, 0.0001 to 0.001 g/L copper, 3.0 to 5.0 g/L yeast extract, 0.15 to 1.5 g/L sucrose, 2.5 to 3.5 g/L dextrose, 1.5 to 3.5 g/L D-trehalose, 0.5 to 1 g/L L-glutamic acid, 0.0001 to 0.0012 g/L L-cysteine, 0.5 to 60 g/L TSB, 0.5 to 1.5 g/L SPS, 0.0004 to 0.0012 g/L menadione, 0.005 to 0.025 g/L Pyridoxal HCl, 0.001 to 0.1 g/L ferulic acid, 0.5 to 3 g/L sodium hydroxide, 0.01 to 0.25 g/L ascorbic acid, and 0.001 to 0.1 g/L hemin, in water.
21. The microbial growth media of any one of the preceding embodiments, wherein the microbial growth media comprises, or comprises about, 0.01 g/L iron; 0.001 g/L copper; 0.528 g/L sucrose; 3.2 g/L dextrose; 3.2 g/L D-trehalose; 4 g/L yeast extract; 0.8 g/L L-glutamic acid; 44 g/L TSB; 0.8 g/L SPS, 0.0008 g/L menadione, 0.016 g/L Pyridoxal HCl; 0.008 g/L ferulic acid; 1.225 g/L sodium hydroxide; 0.08 g/L ascorbic acid; 0.0008 g/L L-cysteine, and 0.008 g/L g hemin.
22. The microbial growth media of any one of the preceding embodiments, wherein the microbial growth media comprises, or comprises about, 0.01 g/L iron; 0.001 g/L copper; 0.33 g/L sucrose; 2 g/L dextrose; 2 g/L D-trehalose; 2.5 g/L yeast extract; 0.5 g/L L-glutamic acid; 27.5 g/L TSB; 0.5 g/L SPS, 0.00005 g/L menadione, 0.01 g/L Pyridoxal HCl; 0.005 g/L ferulic acid; 0.7655 g/L sodium hydroxide; 0.05 g/L ascorbic acid; 0.0005 g/L L-cysteine, and 0.005 g/L hemin.
23. The microbial growth media of any one of the preceding embodiments, wherein the microbial growth media is able to support the growth of one or more microbial species selected from the group consisting of Abiotrophia defectiva, Acinetobacter lwoffii, Agreggatibacter actinomycetemcomitans, Aerococcus viridins, Alcaligenes faecalis, Bacillus subtilis, Candida albicans, Candida glabrata, Cardiobacterium hominis, Corynebacterium jeikeium, Cryptococcus neoformans, Eikinella corrodens, Enterobacter cloacae, Enterococcus faccalis, Escherichia coli, Granulicatella adiacens, Hacmophilus influenzae, Hacmophilus influenzae, Haemophilus influenzae type a, Haemophilus influenzae type b, Haemophilus parainfluenzae, Kingella kingae, Klebsiella pneumoniae, Leuconostoc species, Micrococcus luteus, Neisseria gonorrhoeae, Neisseria meningitidis, Pediococcus acidilactici, Proteus mirabilis, Providencia stuartii, Pseudomonas aeruginosa, Rothia mucilaginosa, Saccharomyces cerevisiae, Staphylococcus aureus, Staphylococcus epidermidis, Stenotrophomonas maltophilia, Streptococcus agalactiae, Streptococcus pneumoniae, Streptococcus pneumoniae, Streptococcus pneumoniae, Streptococcus pneumoniae, Streptococcus pyogenes, and Streptococcus sanguinis.
24. The microbial growth media of any one of the preceding embodiments, wherein the increase in growth rate of Candida is, is about, is at least, is at least about, 10%, 15%, 20%, 25%, 30%, 35%, 40%, 45%, 50%, 60%, 70%, 75%, or 80%,, or is a range defined by any two of the preceding values.
25. The microbial growth media of any one of the preceding embodiments, wherein the reduction in TTD of Candida is, is about, is at least, is at least about, 10%, 15%, 20%, 25%, 30%, 35%, 40%, 45%, 50%, 60%, 70%, 75%, or 80%, or is a range defined by any two of the preceding values.
26. The microbial growth media of any one of the preceding embodiments, wherein the reduction in TTD of Candida is, is about, is at least, is at least about, 1, 2, 3, 4, 5, 6, 7, 8, 9, 10, 12, 15, 18, 24, 30, 36, 48, 60, or 72 hours, or is a range defined by any two of the preceding values.
27. A culture container for detecting growth of a microbe, the container comprising:
   the microbial growth media of any one of the preceding embodiments; and
   a sensor for monitoring a parameter of the microbial growth media indicative of microbial growth in the microbial growth media.
28. The culture container of embodiment 27, wherein the sensor is separated from the contents of the microbial growth media by a permeable membrane.
29. The culture container of embodiment 27 or 28, wherein the parameter monitored is pH, O2, and/or CO2.
30. The culture container of any one of embodiments 27 to 29, wherein the sensor comprises a pH sensor.
31. The culture container of embodiment 30, wherein the pH sensor comprises a fluorescent, phosphorescent, or colorimetric pH responsive agent.
32. The culture container of any one of embodiments 27 to 31, wherein the sensor comprises a O2 sensor.
33. The culture container of embodiment 32, wherein the pH sensor comprises a fluorescent, phosphorescent, or colorimetric O2 responsive agent.
34. The culture container of any one of embodiments 27 to 33, wherein the sensor comprises a CO2 sensor.
35. The culture container of embodiment 34, wherein the CO2 sensor comprises a fluorescent, phosphorescent, or colorimetric pH responsive agent.
36. The culture container of any one of embodiments 27 to 35, wherein the sensor comprises a pH, O2, and/or CO2 sensitive resin.
37. The culture container of embodiment 36, wherein the culture container comprises an amount of pH and/or CO2 sensitive resin that is, is about, is at least, is at least about, is not more than, or is not more than about, 0.01, 0.025, 0.05, 0.075, 0.1, 0.5, 1.0, 2.0, 3.0, 4.0, 5.0, 6.0, 7.0, 8.0, 9.0 or 10.0 g/L, or a range defined by any two of the preceding values.
38. The culture container of any one of embodiments 27 to 37, wherein the container further comprises a headspace volume comprising a gaseous mixture of O2, CO2, and N2.
39. The culture container of embodiment 38, wherein the headspace gas comprises:
   about 25% to about 75% O2,
   about 15% to about 45% CO2, and
   about 0% to about 40% N2.
40. The culture container of embodiment 38 or 39, wherein the headspace gas comprises, comprises about, comprises at least, comprises at least about, comprises less than, or comprises less than about 25%, 30%, 40%, 45%, 46%, 47%, 48%, 49%, 50%, 51%, 52%, 53%, 54%, 55%, 56%, 57%, 58%, 59%, 60%, 65%, 70%, 75% O2, or a range defined by any two of the preceding values.
41. The culture container of embodiment 38 or 39, wherein the headspace gas comprises about 47% to 60% O2.
42. The culture container of embodiment 38 or 39, wherein the headspace gas comprises about 52% to 56% O2.
43. The culture container of any one of embodiments 38 to 42, wherein the headspace gas comprises, comprises about, comprises at least, comprises at least about, comprises less than, or comprises less than about 15%, 20%, 21%, 22%, 23%, 24%, 24.1%, 24.2%, 24.3%, 24.4%, 24.5%, 24.6%, 24.7%, 24.8%, 24.9%, 25%, 26%, 27%, 28%, 29%, 30%, 40%, 45% CO2, or a range defined by any two of the preceding values.
44. The culture container any one of embodiments 38 to 42, wherein the headspace gas comprises about 20% to about 30% CO2.
45. The culture container any one of embodiments 38 to 42, wherein the headspace gas comprises about 24% to about 25% CO2.
46. The culture container of any one of embodiments 36 to 45, wherein the headspace gas comprises, comprises about, comprises at least, comprises at least about, comprises less than, or comprises less than about 20%, 25%, 26%, 27%, 28%, 28.1%, 28.3%, 28.3%, 28.4%, 28.5%, 28.6%, 28.7%,28.8%, 28.9%, 29%, 30%, 31%, 32%, 33%, 34%, 35%, or 40% N2, or a range defined by any two of the preceding values.
47. The culture container of any one of embodiments 38 to 45, wherein the headspace gas comprises about 0% to 30% N2.
48. The culture container of any one of embodiments 38 to 45, wherein the headspace gas consists of, or consists essentially of, O2, CO2, and N2.
49. The culture container of any one of embodiments 27 to 48, wherein the microbial growth is growth of a microbe selected from the group consisting of Abiotrophia defectiva, Acinetobacter lwoffii, Agreggatibacter actinomycetemcomitans, Aerococcus viridins, Alcaligenes faecalis, Bacillus subtilis, Candida albicans, Candida glabrata, Cardiobacterium hominis, Corynebacterium jeikeium, Cryptococcus neoformans, Eikinella corrodens, Enterobacter cloacae, Enterococcus faecalis, Escherichia coli, Granulicatella adiacens, Haemophilus influenzae, Haemophilus influenzae, Haemophilus influenzae type a, Haemophilus influenzae type b, Haemophilus parainfluenzae, Kingella kingae, Klebsiella pneumoniae, Leuconostoc species, Micrococcus luteus, Neisseria gonorrhoeae, Neisseria meningitidis, Pediococcus acidilactici, Proteus mirabilis, Providencia stuartii, Pseudomonas acruginosa, Rothia mucilaginosa, Saccharomyces cerevisiae, Staphylococcus aureus, Staphylococcus epidermidis, Stenotrophomonas maltophilia, Streptococcus agalactiae, Streptococcus pneumoniae, Streptococcus pneumoniae, Streptococcus pneumoniae, Streptococcus pneumoniae, Streptococcus pyogenes, and Streptococcus sanguinis.
50. A system for detecting the presence or absence of a microbe in a sample, the system comprising:
   the culture container of any one of embodiments 27 to 49;
   a detector for obtaining a signal from the sensor;
   a computer configured to determine if the signal obtained by the detector indicates that a microbe is present in the microbial growth media.
51. A method of culturing a microbe in a sample, the method comprising:
   inoculating the microbial growth culture medium of any one of the preceding embodiments with a sample, and culturing a microbe in the sample in the microbial growth culture medium.
52. The method of embodiment 51, wherein the method further comprises detecting the presence or absence of a microbe in the sample.
53. The method of embodiment 51 or 52, wherein the inoculating step comprises adding the sample to the microbial growth culture medium in the culture container of any one of embodiments 27 to 50.
54. The method of embodiment 53, wherein detecting the presence or absence of the microbe in the sample comprises monitoring the sensor for a signal indicative of microbial growth in the microbial growth media.
55. The method of embodiment 54, wherein the signal indicates a change in pH of the microbial growth media.
56. The method of embodiment 54, wherein the signal indicates a change in the CO2 of the culture container headspace gas.
57. The method of any one of embodiments 52 to 55, wherein the microbe is Candida, and the TTD of Candida is reduced as compared to culturing Candida in the same microbial growth media wherein the amount of iron is less than about 0.4 ppm and the amount of copper is less than about 0.04 ppm, or wherein iron and copper are absent from the media.
58. The method of any one of embodiments 51 to 56, wherein said culturing comprises maintaining the microbial culture medium at a temperature of 35° C. to 39° C., or 37° C.
59. The method of any one of embodiments 51 to 58, wherein the method is performed using the system of embodiment 50.
60. The method of any one of embodiments 51 to 59, wherein the sample is selected from the group consisting of, a biological sample, for example, blood, serum, plasma, urine, cerebrospinal fluid, pleural fluid, chest fluid, thoracentesis fluid, peritoneal fluid, abdominal fluid, ascites, pericardial fluid, bone marrow, synovial fluid, or an industrial sample, for example food or pharmaceutical ingredients.
61. The culture container of any one of embodiments 51 to 60, wherein the sample is a biological sample from a systemic inflammatory response syndrome (SIRS)-positive or septic patient.

### BRIEF DESCRIPTION OF THE DRAWINGS

FIG. 1 shows an embodiment of list of organisms tested for improved TTD in the disclosed microbial growth media.

FIG. 2 shows an embodiment of TTD results of Wilcoxon Paired analysis.

FIG. 3 shows an embodiment of results of TTD testing for A. lwoffii in 0, 3, and 10 mL blood samples.

FIG. 4 shows an embodiment of TTD testing for H. parainfluenzae in 0.5, 3, and 10 mL blood samples.

FIG. 5 shows an embodiment of results of TTD testing for three additional strains of H. parainfluenzae in 0.5, 3, and 10 mL blood samples.

FIG. 6 shows an embodiment of results of TTD testing for N. meningitidis in 0.5, 3, and 10 mL blood samples.

FIG. 7 shows an embodiment of results of TTD testing for R. mucilaginosa in 0, 3, and 10 mL blood samples.

FIG. 8 shows an embodiment of results of TTD testing for three additional strains of R. mucilaginosa in 0, 3, and 10 mL blood samples.

FIG. 9 shows an embodiment of results of TTD testing for S. maltophilia in 0, 3, and 10 mL blood samples.

FIG. 10 shows an embodiment of results of TTD testing for five additional strains of R. mucilaginosa in 0, 3, and 10 mL blood samples.

FIG. 11 shows an embodiment of results of TTD testing for S. pneumoniae in 0, 3, and 10 mL blood samples.

FIG. 12 shows an embodiment of results of repeated TTD testing for S. pneumoniae in 0, 3, and 10 mL blood samples

FIG. 13 shows an embodiment of a list of organisms used in percent recovery testing.

FIG. 14 shows an embodiment of results of percent recovery testing in BD Plus Aerobic/26F BACTEC (PFS) vs the disclosed microbial growth media (SJ) using a McNemar's Chi Square test.

FIG. 15 shows an embodiment of results of false positive testing.

FIG. 16 shows an embodiment of a list of organisms used in DVE testing.

FIG. 17 shows an embodiment of DVE testing in BD Plus Aerobic/26F BACTEC (PFS) as compared to the disclosed microbial growth media (SJ).

FIG. 18 shows an embodiment of a table of organisms, antibiotics, and antibiotic concentrations used in growth support testing.

FIG. 19 shows an embodiment of results of growth support testing in in BD Plus Aerobic/26F BACTEC (PFS) vs the disclosed microbial growth media (SJ) using a McNemar's Chi Square test.

FIG. 20 shows an embodiment of a table of GPC, GNB, Yeast, Neisseria/Haemophilus, and GP/GNCB groups of microorganisms and representative microorganisms belong to each class.

FIG. 21 shows an embodiment of a table of the combinations of iron concentration and O₂ concentration tested for improved TTD.

FIG. 22 shows an embodiment of histograms and quantification of the TTD of microorganisms in blood volumes of 0, 0.5, 3, or 10 mL.

FIG. 23 shows an embodiment of histograms of the TTD of GC/Haem, GNB, GPB/GNCB, GPC, and Yeast groups, in microbial growth media bottles comprising 47%, 54%, and 60% O₂ in the headspace gas.

FIG. 24 shows an embodiment of histograms of the TTD of GC/Haem, GNB, GPB/GNCB, GPC, and Yeast groups, in microbial growth media comprising 0, 0.01, 0.035, and 0.06 g/L iron.

FIG. 25 shows an embodiment of dot plots of the TTD of GC/Haem, GNB, GPB/GNCB, GPC, and Yeast groups, in microbial growth media comprising 0, 0.01, 0.035, and 0.06 g/L iron.

FIG. 26 shows an embodiment of dot plots of the TTD of GC/Haem, GNB, GPB/GNCB, GPC, and Yeast groups, in microbial growth media comprising 47%, 54%, and 60% O₂ in the headspace gas.

FIG. 27 shows an embodiment of dot plots of the TTD of various microorganisms in microbial growth media comprising 0, 0.01, 0.035, and 0.06 g/L iron.

FIG. 28 shows an embodiment of dot plots of the TTD of various microorganisms in microbial growth media bottles comprising 47%, 54%, and 60% O₂ in the headspace gas.

FIG. 29 shows an embodiment of histograms of the TTD in yeast in microbial growth media comprising 0, 0.01, 0.035, and 0.06 g/L iron.

FIG. 30 shows an embodiment of histograms of the combined TTD in GPC, GNB, Neisseria/Haemophilus, and GP/GNCB groups of microorganisms in microbial growth media comprising 0, 0.01, 0.035, and 0.06 g/L iron.

FIG. 31 shows an embodiment of histograms illustrating the improved TTD in yeast as compared to the TTD in GPC, GNB, Neisseria/Haemophilus, and GP/GNCB groups of microorganisms.

FIG. 32 shows an embodiment of bivariate fits of the TTD of microorganisms in the disclosed microbial growth media bottles (ICE Control Standard) with a headspace gas comprising 47% O₂.

FIG. 33 shows an embodiment of summary histogram and Wilcoxon paired analysis data of the TTD of microorganisms in the disclosed microbial growth media bottles (ICE Control Standard) with a headspace gas comprising 47% O₂.

FIG. 34 shows an embodiment of bivariate fits of the TTD of microorganisms in the disclosed microbial growth media bottles (ICE Control 54) with a headspace gas comprising 54% O₂.

FIG. 35 shows an embodiment of summary histogram and Wilcoxon paired analysis data of the TTD of microorganisms in the disclosed microbial growth media bottles (ICE Control 54) with a headspace gas comprising 54% O₂.

FIG. 36 shows an embodiment of bivariate fits of the TTD of microorganisms in the disclosed microbial growth media bottles (ICE Control 60) with a headspace gas comprising 54% O₂.

FIG. 37 shows an embodiment of summary histogram and Wilcoxon paired analysis data of the TTD of microorganisms in the disclosed microbial growth media bottles (ICE Control 54) with a headspace gas comprising 60% O₂.

FIG. 38 shows an embodiment of bivariate fits of the TTD of microorganisms in the disclosed microbial growth media bottles containing microbial growth media comprising 0.01 g/L iron and a gaseous headspace comprising 47% O₂ (ICE 0.01 Fe Standard).

FIG. 39 shows an embodiment of a summary histogram and Wilcoxon paired analysis data of the TTD of microorganisms in the disclosed microbial growth media bottles containing microbial growth media comprising 0.01 g/L iron and a gaseous headspace comprising 47% O₂ (ICE 0.01 Fe Standard).

FIG. 40 shows an embodiment of bivariate fits of the TTD of microorganisms in the disclosed microbial growth media bottles containing microbial growth media comprising 0.035 g/L iron and a gaseous headspace comprising 47% O₂ (ICE 0.035 Fe Standard).

FIG. 41 shows an embodiment of a summary histogram and Wilcoxon paired analysis data of the TTD of microorganisms in the disclosed microbial growth media bottles containing microbial growth media comprising 0.035 g/L iron and a gaseous headspace comprising 47% O₂ (ICE 0.035 Fe Standard).

FIG. 42 shows an embodiment of bivariate fits of the TTD of microorganisms in the disclosed microbial growth media bottles containing microbial growth media comprising 0.06 g/L iron and a gaseous headspace comprising 47% O₂ (ICE 0.06 Fe Standard).

FIG. 43 shows an embodiment of a summary histogram and Wilcoxon paired analysis data of the TTD of microorganisms in the disclosed microbial growth media bottles containing microbial growth media comprising 0.06 g/L iron and a gaseous headspace comprising 47% O₂ (ICE 0.06 Fc Standard).

FIG. 44 shows an embodiment of bivariate fits of the TTD of microorganisms in the disclosed microbial growth media bottles containing microbial growth media comprising 0.01 g/L iron and a gaseous headspace comprising 54% O₂ (ICE 0.01 Fe 54).

FIG. 45 shows an embodiment of a summary histogram and Wilcoxon paired analysis data of the TTD of microorganisms in the disclosed microbial growth media bottles containing microbial growth media comprising 0.01 g/L iron and a gaseous headspace comprising 54% O₂ (ICE 0.01 Fe 54.

FIG. 46 shows an embodiment of bivariate fits of the TTD of microorganisms in the disclosed microbial growth media bottles containing microbial growth media comprising 0.35 g/L iron and a gaseous headspace comprising 54% O₂ (ICE 0.035 Fe 54).

FIG. 47 shows an embodiment of a summary histogram and Wilcoxon paired analysis data of the TTD of microorganisms in the disclosed microbial growth media bottles containing microbial growth media comprising 0.035 g/L iron and a gaseous headspace comprising 54% O₂ (ICE 0.035 Fe 54.

FIG. 48 shows an embodiment of bivariate fits of the TTD of microorganisms in the disclosed microbial growth media bottles containing microbial growth media comprising 0.06 g/L iron and a gaseous headspace comprising 54% O₂ (ICE 0.06 Fe 54).

FIG. 49 shows an embodiment of a summary histogram and Wilcoxon paired analysis data of the TTD of microorganisms in the disclosed microbial growth media bottles containing microbial growth media comprising 0.06 g/L iron and a gaseous headspace comprising 54% O₂ (ICE 0.06 Fe 54).

FIG. 50 shows an embodiment of bivariate fits of the TTD of microorganisms in the disclosed microbial growth media bottles containing microbial growth media comprising 0.01 g/L iron and a gaseous headspace comprising 60% O₂ (ICE 0.01 Fe 60).

FIG. 51 shows an embodiment of a summary histogram and Wilcoxon paired analysis data of the TTD of microorganisms in the disclosed microbial growth media bottles containing microbial growth media comprising 0.01 g/L iron and a gaseous headspace comprising 60% O₂ (ICE 0.01 Fe 60).

FIG. 52 show representative bivariate fits of the TTD of microorganisms in the disclosed microbial growth media bottles microbial growth media comprising 0.035 g/L iron and a gaseous headspace comprising 60% O₂ (ICE 0.035 Fe 60).

FIG. 53 shows an embodiment of a summary histogram and Wilcoxon paired analysis data of the TTD of microorganisms in the disclosed microbial growth media bottles containing microbial growth media comprising 0.035 g/L iron and a gaseous headspace comprising 60% O₂ (ICE 0.035Fe 60).

FIG. 54 shows an embodiment of bivariate fit embodiments of the TTD of microorganisms in the disclosed microbial growth media bottles containing microbial growth media comprising 0.06 g/L iron and a gaseous headspace comprising 60% O₂ (ICE 0.06 Fe 60).

FIG. 55 shows an embodiment of a summary histogram and Wilcoxon paired analysis data of the TTD of microorganisms in the disclosed microbial growth media bottles containing microbial growth media comprising 0.06 g/L iron and a gaseous headspace comprising 60% O₂ (ICE 0.06 Fe 60).

FIG. 56 shows an embodiment of ANOVA results of the effects of varying concentrations of iron and O₂ on the TTD of microorganisms in microbial growth media.

FIG. 57 shows an embodiment of ANOVA results of the parameter estimates and prediction equation for the TTD of the GC/Haem, GNB, GPB/GNCB, GPC, and yeast groups of microorganisms in microbial growth media.

### DETAILED DESCRIPTION

Although certain embodiments and examples are disclosed below, inventive subject matter extends beyond the specifically disclosed embodiments to other alternative embodiments and/or uses and to modifications and equivalents thereof. Thus, the scope of the presently disclosed invention is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

The early detection of bacterial and fungal infections is of paramount importance to guide antimicrobial therapy in septic patients. Sepsis is a potentially life-threatening condition that occurs when a subjects body damages its own tissues in response to infection. Sepsis may progress to septic shock, a dramatic drop in the subject's blood pressure that may result in severe organ damage and death. As of 2013, there were approximately 20,000 sepsis related deaths per day, worldwide. This equates to an estimated 18,000,000 deaths per year. Between 2000 and 2008, the number of U.S. hospital admissions for sepsis more than doubled. In 2009, sepsis was the most expensive reason for hospitalization in the U.S., totaling nearly $15.4 billion USD in aggregate hospital costs. This increased hospitalization cost yielded little benefit to the septic subject as, in 2009, U.S. mortality rates were eight times higher than mortality rates form other hospital stays. Each hour delay in administration of effective antimicrobials can increase mortality rates by 7.6%. microbial growth media testing allows for identification of microorganisms in a subject with a systemic infection. Current microbial growth media is comprised of unspecified amounts of iron and copper in addition to other nutrients and often have long time to detection (TTD) of microorganisms in biological samples.

In some embodiments, the present disclosure is related to an microbial growth media with improved growth rate and/or TTD of microorganisms. In some embodiments, the microorganism is Candida. In some embodiments, the microorganism is Candida albicans or Candida glabrata. In some embodiments the microorganism is in a biological sample. In some embodiments, the biological sample is blood. In some embodiments, the biological sample is scrum or plasma. In some embodiments, the biological sample is urine. In some embodiments, the biological sample is a bodily fluid which is sterile in a healthy patient, for example, blood, urine, cerebrospinal fluid, pleural fluid, chest fluid, thoracentesis fluid, peritoneal fluid, abdominal fluid, ascites, pericardial fluid, bone marrow, synovial fluid. In some embodiments the biological sample is obtained from a septic subject. In some embodiments the biological sample is obtained from a systemic inflammatory response syndrome (SIRS)-positive subject. In some embodiments, the subject has a systemic infection. In some embodiments, the systemic infection is a bacterial infection. In some embodiments, the bacteria is a gram negative bacterium. In some embodiments, the bacterium is a gram positive bacterium. In some embodiments, the infection is a yeast infection. In some embodiments, the infection is an Abiotrophia defectiva, Acinetobacter lwoffii, Agreggatibacter actinomycetemcomitans, Aerococcus viridins, Alcaligenes faecalis, Bacillus subtilis, Candida albicans, Candida glabrata, Cardiobacterium hominis, Corynebacterium jeikeium, Cryptococcus neoformans, Eikinella corrodens, Enterobacter cloacae, Enterococcus faecalis, Escherichia coli, Granulicatella adiacens, Haemophilus influenzae, Haemophilus influenzae, Haemophilus influenzae type a, Haemophilus influenzae type b, Haemophilus parainfluenzae, Kingella kingae, Klebsiella pneumoniae, Leuconostoc species, Micrococcus luteus, Neisseria gonorrhoeae, Neisseria meningitidis, Pediococcus acidilactici, Proteus mirabilis, Providencia stuartii, Pseudomonas aeruginosa, Rothia mucilaginosa, Saccharomyces cerevisiae, Staphylococcus aureus, Staphylococcus epidermidis, Stenotrophomonas maltophilia, Streptococcus agalactiae, Streptococcus pneumoniae, Streptococcus pneumoniae, Streptococcus pneumoniae, Streptococcus pneumoniae, Streptococcus pyogenes, or Streptococcus sanguinis infection. In some embodiments the sample is from an industrial application where the sample is tested for sterility, for example, a sample of a food or pharmaceutical product, e.g., for USP applications.

In some embodiments, the microbial growth media comprises iron. In some embodiments the iron is present as ferric iron (Fe(III) or Fe³⁺). In some embodiments, the iron is provided as ferrous iron (Fe(II) or Fe²⁺). In some embodiments, the iron comprises an iron salt or an iron complex. In some embodiments, the iron is provided as ferric ammonium citrate, ferric chloride, ferric sulfate, ferric nitrate, and/or another suitable ferric iron salt or complex. In some embodiments, the iron is provided as the corresponding ferrous iron salt or complex of the forgoing. In some embodiments, the amount of iron is, is about, is at least, is at least about, is not more than, or is not more than about, 1, 2, 3, 4, 5, 6, 7, 8, 9, 10, 15, 20, 25, 28, 50, 75, 100, 125, 150, 170, 175, 200, 225, 250, 275, 300, 325, 350, 375, or 400 μM, or is a range defined by any two of the preceding values. For example, in some embodiments, the amount of iron is, or is about: 1 to 400 μM, 1 to 350 μM, 1 to 200 μM, 1 to 100 μM, 1 to 50 μM, 2 to 400 μM, 2 to 350 μM, 25 to 400 μM, 25 to 325 μM, 25 to 200 μM, 25 to 100 μM, or 25 to 75 μM. In some embodiments, the amount of iron is, is about, is at least, is at least about, is not more than, or is not more than about, 28, 100 or 170 μM. In some embodiments, the amount of iron is, or is about, 28 to 170 μM. In some embodiments, the amount of iron is, is about, is at least, is at least about, is not more than, or is not more than about, 1, 1.5, 2, 2.5, 3, 3.5, 4, 4.5, 5, 5.5, 6, 6.5, 7, 7.5, 8, 8.5, 9, 9.5, or 10 ppm, or is in a range that is defined by any two of the preceding values. For example, in some embodiments, the amount of iron is, or is about: 1 to 10 ppm, 1 to 7.5 ppm, 1 to 5 ppm, 2.5 to 10 ppm, 2.5 to 7.5, ppm, 2.5 to 5 ppm, 5 to 10 ppm, or 5 to 7.5 ppm.

In some embodiments, the microbial growth media comprises copper. In some embodiments, the copper is present as cupric copper (Cu(II) or Cu²⁺). In some embodiments, the copper comprises a copper salt or a copper complex. In some embodiments, the copper is provided as cupric sulfate, cupric chloride, cupric nitrate, cupric bromide, cupric chlorate, copper(II) gluconate, or another suitable copper salt or complex. In some embodiments, the amount of copper is, is about, is at least, is at least about, is not more than, or is not more than about, 0, 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 75, 100, 150, 200, 300, 400, 500, 600, 650, or 700 μM copper, or is a range defined by any two of the preceding values. For example, in some embodiments, the amount of copper is, or is about, 0 to 700 μM, 0 to 650 μM, 0 to 500 μM, 0 to 300 μM, 0 to 100 μM, 0 to 50 μM, 0 to 25 μM, 0.1 to 100 μM, 0.1 to 50 μM, 0.1 to 25 μM, 0.5 to 650 μM, 0.5 to 300 μM, 0.5 to 100 μM, 0.5 to 50 μM, or 0.5 to 25 μM. In some embodiments, the amount of copper is, is about, is at least, is at least about, is not more than, or is not more than about, 0.05, 0.075, 0.1, 0.2, 0.3, 0.4, or 0.5 ppm, or is in a range that is defined by any two of the preceding values. For example, in some embodiments, the amount of iron is, or is about: 0.05 to 0.5 ppm, 0.05 to 0.3 ppm, 0.05 to 2 ppm, 0.1 to 0.5 ppm, or 0.1 to 0.3 ppm.

In some embodiments the enhanced microbial growth media comprises yeast nitrogen base. In some embodiments, the yeast nitrogen base is lacking amino acids. In some embodiments, the yeast nitrogen base is lacking histidine, methionine, tryptophan, and/or any combination therein. In some embodiments, the yeast nitrogen base is lacking amino acids other than histidine, methionine, and/or tryptophan. In some embodiments, the yeast nitrogen base, with or without certain amino acids, is omitted from or is not present in the microbial growth media. In some embodiments, omission of the yeast nitrogen base from the microbial growth media minimizes precipitation of iron out of solution. In some embodiments yeast nitrogen base is present in an amount that is less than an amount that causes the iron in the microbial growth media to precipitate out of solution. In some embodiments, the amount of yeast nitrogen base is, is about, is at least, is at least about, is not more than, or is not more than about, 0, 0.01, 0.1, 0.2, 0.3, 0.4, 0.5, 0.6, 0.7, 0.8, 0.9, or 1.0 g/L yeast nitrogen base, or is a range defined by any two of the preceding values. For example, in some embodiments, the amount of yeast nitrogen base is, or is about, 0.00 to 1 g/L, 0.00 to 0.8 g/L, 0.00 to 0.4 g/L, 0.00 to 0.1 g/L, 0.1 to 1 g/L, 0.1 to 0.8 g/L, or 0.1 to 0.4 g/L yeast nitrogen base.

In some embodiments, the microbial growth media comprises yeast extract. In some embodiments, the amount of yeast extract is, is about, is at least, is at least about, is not more than, or is not more than about, 0.0, 1.0, 1.5, 2.0, 2.5, 3.0, 3.5, 3.6, 3.7, 3.8. 3.9, 4.0, 4.1, 4.2, 4.3, 4.4, 4.5, 5.0, 6.0, 7.0, 8.0, 9.0, or 10.0 g/L yeast extract, or is a range defined by any two of the preceding values. For example, in some embodiments, the amount of yeast extract is, or is about 1.0 to 10.0 g/L, 1.0 to 4.0 g/L, 2.5 to 10.0 g/L, or 2.5 to 4.0 g/L.

In some embodiments, the microbial growth media comprises one or more carbohydrates. In some embodiments, the carbohydrate comprises sucrose. In some embodiments, the carbohydrate comprises glucose, fructose, or another suitable carbohydrate. In some embodiments, the amount of sucrose is, is about, is at least, is at least about, is not more than, or is not more than about, 0.15, 0.25, 0.30, 0.31, 0.32, 0.33, 0.34, 0.35, 0.40, 0.45, 0.46, 0.47, 0.48, 0.49, 0.50, 0.51, 0.52, 0.53, 0.54, 0.55, 0.6, 0.75, or 1.0g/L sucrose, or is a range defined by any two of the preceding values. For example, in some embodiments, the amount of sucrose is, or is about, 0.15 to 1.0 g/L, 0.30 to 1.0 g/L, or 0.30 to 0.60 g/L. In some embodiments, the carbohydrate comprises dextrose. In some embodiments, the microbial growth media comprises between, or between about, 1.0 and 10.0 g/L dextrose. In some embodiments, the amount of dextrose is, is about, is at least, is at least about, is not more than, or is not more than about, 1.0, 1.5, 2.0, 2.5, 2.6, 2.6, 2.8, 2.9, 3.0, 3.1, 3.2, 3.3, 3.4, 3.5, 3.6, 3.7, 4.0, 4.5, 5.0, 6.0, 7.0, 8.0, 9.0, or 10.0 g/L dextrose, or is a range defined by any two of the preceding values. For example, in some embodiments, the amount of dextrose is 1.0 to 10.0 g/L, 1.0 to 5.0 g/L, 1.0 to 3.2 g/L, 2.0 to 10.0 g/L, or 2.0 to 3.2 g/L. In some embodiments, the microbial growth media comprises both sucrose and dextrose. In some embodiments, the microbial growth media comprises either sucrose or dextrose. In some embodiments, carbohydrate comprises D-trehalose. In some embodiments, about the amount of D-trehalose is, is about, is at least, is at least about, is not more than, or is not more than about, 1.0, 1.5, 2.0, 2.5, 2.6, 2.6, 2.8, 2.9, 3.0, 3.1, 3.2, 3.3, 3.4, 3.5, 3.6, 3.7, 4.0, 4.5, 5.0, 6.0, 7.0, 8.0, 9.0, or 10.0 g/L D-trehalose, or is a range defined by any two of the preceding values. For example, in some embodiments, the amount of D-trehalose is, or is about, 1.0 to 10.0 g/L, 1.0 to 5.0 g/L, 1.0 to 3.2 g/L, 2.0 to 10.0 g/L, or 2.0 to 3.2 g/L. In some embodiments, the microbial growth media comprises sucrose, dextrose, and/or D-trehalose. In some embodiments, another suitable carbohydrate is substituted for sucrose, dextrose, and/or D-trehalose, e.g., glucose. In some embodiments, the concentration of the carbohydrate in the microbial growth media is chosen specifically for that concentration's suitability for use in supporting the growth of one or more microorganisms.

In some embodiments, the microbial growth media comprises one or more amino acids. In some embodiments, the amino acid is isoleucine, leucine, valine, histidine, lysine, methionine, phenylalanine, threonine, tryptophan, citrulline, GABA, hydroxyproline, oxoproline, ornithine, asparagine, aspartic acid, aspartate, alanine, arginine, cysteine, L-cysteine, cystine, glutamine, glutamic acid, L-glutamic acid, glutamate, glycine, proline, serine, tyrosine, acetlyhydroxyproline, alanyl-glutamine, and/or glutathione. In some embodiments, the amino acid is L-glutamic acid. In some embodiments, the amount of L-glutamic acid is, is about, is at least, is at least about, is not more than, or is not more than about, 0.25, 0.5, 0.6, 0.7, 0.8, 0.9, 1.0, 1.1, 1.2, or 1.5g/L L-glutamic acid, or is a range defined by any two of the preceding values. For example, in some embodiments, the amount of L-glutamic acid is, or is about, 0.25 to 1.5 g/L, 0.25 to 1.0 g/L, 0.25 to 0.8 g/L, 0.25 to 0.5 g/L, 0.5 to 1.5 g/L, or 0.5 to 0.8 g/L. In some embodiments, the amino acid is L-cysteine. In some embodiments, the amount of L-cysteine is, is about, is at least, is at least about, is not more than, or is not more than about, 0.0001, 0.0002, 0.0003, 0.0004, 0.0005, 0.0006, 0.007, 0.0008, 0.0009 or 0.001 g/L L-cysteine, or is a range defined by any two of the preceding values. For example, in some embodiments, the amount of L-cysteine is, or is about, 0.0001 to 0.001 g/L, 0.0001 to 0.0008 g/L, 0.0005 to 0.001 g/L, or 0.0005 to 0.0008 g/L. In some embodiments, the microbial growth media comprises both L-glutamic acid and L-cysteine. In some embodiments, the amino acids and/or concentration of amino acids in the microbial growth media are chosen for their suitability in promoting the growth of one or more specific microorganisms.

In some embodiments, the microbial growth media comprises tryptic soy broth (TSB). In some embodiments, about the amount of TSB is, is about, is at least, is at least about, is not more than, or is not more than about, 15, 20, 25, 27.5, 30, 35, 40, 44, 45, 50, 55, or 60 g/L TSB, or is a range defined by any two of the preceding values. For example, in some embodiments, the amount of TSB is, or is about, 15 to 60 g/L, 15 to 44 g/L, 15 to 27.5 g/L, 27.5 to 60 g/L, or 27.5 to 44 g/L.

In some embodiments, the microbial growth media comprises sodium polyanethole sulfonate (SPS). In some embodiments, the amount of SPS is, is about, is at least, is at least about, is not more than, or is not more than about, 0.25, 0.5, 0.6, 0.7, 0.8, 0.9, 1.0, 1.1, 1.2, or 1.5 g/L SPS, or is a range defined by any two of the preceding values. For example, in some embodiments, the amount of SPS is, or is about, 0.25 to 1.5 g/L, 0.25 to 1.0 g/L, 0.25 to 0.8 g/L, 0.5 to 1.5 g/L, or 0.5 to 0.8 g/L.

In some embodiments, the microbial growth media comprises menadione. In some embodiments, the amount of menadione is, is about, is at least, is at least about, is not more than, or is not more than about 0.001, 0.002, 0.003, 0.004, 0.005, 0.006, 0.007, 0.008, 0.009 or 0.01 g/L menadione, or is a range defined by any two of the preceding values. For example, in some embodiments, the amount of menadione is, or is about, 0.001 to 0.01 g/L, 0.001 to 0.008 g/L, 0.005 to 0.01 g/L, or 0.005 to 0.008 g/L.

In some embodiments, the microbial growth media comprises Pyridoxal HCl. In some embodiments, the amount of Pyridoxal HCl is, is about, is at least, is at least about, is not more than, or is not more than about, 0.005 0.010, 0.011, 0.012, 0.013, 0.014, 0.015, 0.016, 0.017, 0.018, 0.019, 0.020, 0.025, or 0.030 1 g/L Pyridoxal HCl, or is a range defined by any two of the preceding values. For example, in some embodiments, the amount of Pyridoxal HCl is, or is about 0.005 to 0.03 g/L, 0.01 to 0.03 g/l, 0.01 to 0.02 g/L, or 0.01 to 0.016 g/L.

In some embodiments, the microbial growth media comprises ferulic acid. In some embodiments, the amount of ferulic acid is, is about, is at least, is at least about, is not more than, or is not more than about, 0.010, 0.020, 0.030, 0.040, 0.050, 0.06, 0.07, 0.08, 0.09, 0.1, or 0.15 g/L ferulic acid, or is a range defined by any two of the preceding values. For example, in some embodiments, the amount of ferulic acid is, or is about, 0.010 to 0.15 g/L, 0.01 to 0.1 g/L, 0.01 to 0.08 g/L 0.05 to 0.15 g/L, or 0.05 to 0.08 g/L.

In some embodiments, the microbial growth media comprises sodium hydroxide. In some embodiments, the amount of sodium hydroxide is, is about, is at least, is at least about, is not more than, or is not more than about, 0.5, 0.6, 0.7, 0.8, 0.9, 1.0, 1.1, 1.2, 1.3, 1.4, or 1.5 g/L sodium hydroxide, or is a range defined by any two of the preceding values. For example, in some embodiments, the amount of sodium hydroxide is, or is about, 0.5 to 1.5 g/L, 0.5 to 1.225 g/L, 0.5 to 1.0 g/L, 0.75 to 1.5 g/L, or 0.75 to 1.225 g/L sodium hydroxide.

In some embodiments, the microbial growth media comprises an anti-oxidant. In some embodiments, the anti-oxidant comprises ascorbic acid. In some embodiments, the amount of ascorbic acid is, is about, is at least, is at least about, is not more than, or is not more than about, 0.025, 0.03, 0.04, 0.05, 0.06, 0.07, 0.08, 0.09, 0.10, or 0.15 g/L ascorbic acid, or is a range defined by any two of the preceding values. For example, in some embodiments, the amount of ascorbic acid is, or is about, 0.025 to 0.15 g/L, 0.025 to 0.10 g/L, 0.025 to 0.08 g/L, 0.05 to 0.15 g/L, 0.05 to 0.10 g/L, or 0.05 to 0.08 g/L.

In some embodiments, the microbial growth media comprises hemin. In some embodiments, the amount of hemin is, is about, is at least, is at least about, is not more than, or is not more than about, 0.010, 0.020, 0.030, 0.040, 0.050, 0.06, 0.07, 0.08, 0.09, 0.1, or 0.15 g/L hemin, or is a range defined by any two of the preceding values. For example, in some embodiments, the amount of hemin is, or is about, 0.010 to 0.15 g/L, 0.01 to 0.1 g/L, 0.01 to 0.08 g/L 0.05 to 0.15 g/L, or 0.05 to 0.08 g/L.

In some embodiments, the microbial growth media comprises water, e.g., deionized (DI) water, iron, copper, sucrose dextrose D-trehalose, yeast extract, L-glutamic acid, tryptic soy broth (TSB), sodium polyanethole sulfonate (SPS), menadione, pyridoxal HCl, ferulic acid, sodium hydroxide, ascorbic acid, L-cysteine, and hemin. In some embodiments, the microbial growth medium comprises, or comprises about, 0.001 g/L to 0.15 g/L iron, e.g. ferric iron (e.g., provided as ferric ammonium citrate), 0.0001 to 0.0020 g/L copper, e.g. cupric copper (e.g., provided as cupric sulfate), 1.0 to 10.0 g/L yeast extract, 0.15 to 1.0 g/L sucrose, 1.0 to 10.0 g/L dextrose, 1.0 to 10.0 g/L D-trehalose, 0.001 to 0.01 g/L L-glutamic acid, 0.0001 to 0.001 g/L L-cysteine, 15 to 60 g/L TSB, 0.25 to 1.5 g/L SPS, 0.001 to 0.01 g/L menadione, 0.005 to 0.030 g/L Pyridoxal HCl, 0.010 to 0.15 g/L ferulic acid, 0.5 to 1.5 g/L sodium hydroxide, 0.025 to 0.15 g/L ascorbic acid, 0.01 to 0.15 g/L hemin. In some embodiments, the microbial growth medium comprises water (e.g. DI water); 0.01 g/L to 0.06 g/L iron, e.g. ferric iron (e.g., provided as ferric ammonium citrate); 0.0005 to 0.0015 g/L copper, e.g. cupric copper (e.g., provided as cupric sulfate); 2.5 to 4.0 g/L yeast extract; 0.35 to 0.6 g/L sucrose; 2.0 to 3.2 g/L dextrose; 2.0 to 3.2 g/L D-trehalose; 0.5 to 0.8 g/L L-glutamic acid; 0.0005 to 0.0008 g/L L-cysteine; 2.5 to 44 g/L TSB; 0.5 to 0.8 g/L SPS; 0.005 to 0.008 g/L menadione; 0.010 to 0.016 g/L Pyridoxal HCl; 0.05 to 0.08 g/L ferulic acid; 0.75 to 1.225 g/L sodium hydroxide; 0.05 to 0.08 g/L ascorbic acid; 0.05 to 0.08 g/L hemin. In some embodiments, the microbial growth media comprises, or comprises about, 0.01 g/L iron; 0.001 g/L copper; 0.528 g/L sucrose; 3.2 g/L dextrose; 3.2 g/L D-trehalose; 4 g/L yeast extract; 0.8 g/L L-glutamic acid; 44 g/L TSB; 0.8 g/L SPS, 0.0008 g/L menadione, 0.016 g/L Pyridoxal HCl; 0.008 g/L ferulic acid; 1.225 g/L sodium hydroxide; 0.08 g/L ascorbic acid; 0.0008 g/L L-cysteine, and 0.008 g/L g hemin. In some embodiments, the bacterial growth media comprises, or comprises about, 0.01 g/L iron; 0.001 g/L copper; 0.33 g/L sucrose; 2 g/L dextrose; 2 g/L D-trehalose; 2.5 g/L yeast extract; 0.5 g/L L-glutamic acid; 27.5 g/L TSB; 0.5 g/L SPS, 0.00005 g/L menadione, 0.01 g/L Pyridoxal HCl; 0.005 g/L ferulic acid; 0.7655 g/L sodium hydroxide; 0.05 g/L ascorbic acid; 0.0005 g/L L-cysteine, and 0.005 g/L hemin.

In some embodiments, the present disclosure is related to a microbial growth media with improved growth rate and/or TTD of Candida e.g., Candida albicans or Candida glabrata, other yeast and/or other microorganisms. In some embodiments the microorganism is in a biological sample. In some embodiments, the biological sample is a blood, serum, plasma, or urine sample. In some embodiments the biological sample is obtained from a septic subject. In some embodiments the biological sample is obtained from a systemic inflammatory response syndrome (SIRS)-positive subject. In some embodiments, the addition of iron and/or copper to the microbial growth media as disclosed herein reduces the TTD of Candida e.g., Candida albicans or Candida glabrata, other yeast and/or other microorganisms in a biological sample added to the growth media. In some embodiments, the addition of iron and/or copper to the microbial growth media as disclosed herein reduces the TTD of Candida e.g., Candida albicans or Candida glabrata, other yeast, and/or other microorganisms in a blood, serum, plasma, or urine sample added to the microbial growth media. In some embodiments, the microbial growth media has similar percent recovery, delayed vial entry (DVE), false positive rate, and growth support with antimicrobials as the same microbial growth media without the iron and copper content disclosed herein. In some embodiments, the microbial growth media has enhanced percent recovery, DVE, false positive rate, growth support with anti-microbials, or any combination thereof, as compared to the same growth media without the iron and copper content disclosed herein. In some embodiments, the addition of iron and/or copper to the microbial growth media as disclosed herein increases the growth rate of Candida e.g., Candida albicans or Candida glabrata, other yeast and/or other microorganisms. In some embodiments, the microbial growth media has, has about, has at least, has at least about, has not more than, or has not more than about, a 10%, 15%, 20%, 25%, 30%, 40%, 45%, 50%, 60%, 70%, 75%, or 80% increase in the growth rate of microorganisms (e.g., Candida e.g., Candida albicans or Candida glabrata) in a biological sample, or is a range defined by any two of the preceding values. For example, in some embodiments, the increased microbial growth rate is, or is about, a 10% to 80%, 10% to 50%, 10% to 20%, 25% to 80%, or 25% to 50% increase. In some embodiments, the microbial growth media has, has about, has at least, has at least about, has not more than, or has not more than about, a 10%, 15%, 20%, 25%, 30%, 40%, 45%, 50%, 60%, 70%, 75%, or 80% reduction in the TTD of microorganisms (e.g., Candida e.g., Candida albicans or Candida glabrata) in a biological sample, or the TTD is reduced by a range defined by any two of the preceding values. For example, in some embodiments, the TTD reduction is, or is about, a 10% to 80%, 10% to 50%, 10% to 20%, 25% to 80%, or 25% to 50% reduction. In some embodiments, the TTD of microorganisms in the microbial growth media is reduced by, by about, by at least, by about at least, by not more than, or by not more than about, 1, 2, 3, 4, 5, 6, 7, 8, 9, 10, 12, 15, 18, 24, 30, 36, 48, 60, or 72 hours, or the TTD is reduced by a range defined by any two of the preceding values. For example, in some embodiments, the TTD is reduced by, or by about, 0 to 72 hours.

In some embodiments, the reduced TTD of Candida (e.g., Candida e.g., Candida albicans or Candida glabrata) cultured in the microbial growth media is determined by amending culture containers (e.g., BD BACTEC blood culture bottles, Becton, Dickinson and Co.) comprising 30 ml of microbial growth media with and without the iron and/or copper with 0.5, 3, or 10 mL of blood (or optionally another biological sample (e.g., serum, saliva, or urine) and inoculating with 0.1 mL of Candida, at a final target concentration of 10 to 100 CFU, e.g., 50 CFU, per bottle. The Candida is then cultured at about 30° C. to 37° C. for up to about, 120 hours. The culture container is monitored for a signal (e.g. fluorescence) indicative of the presence of Candida (e.g. change in pH and/or CO₂) and the TTD of the Candida in the microbial growth media with and without the iron and/or copper is determined. In some embodiments, the median of the interval taken for receipt of the signal indicative of the presence of Candida in the paired culture containers is compared. In some embodiments, the culturing, detection the presence of Candida and determination of TTD is performed on an automated instrument, for example the BACTEC FX instrument (Becton, Dickinson and Co.). In some embodiments, paired bottles with and without the iron and/or copper are entered into the instrument at the same time in adjacent positions on the instrument.

In some embodiments, a culture container for detecting growth of a microbe is provided. In some embodiments, the microbial organism comprises Candida e.g., Candida albicans or Candida glabrata, other yeast and/or other microorganisms. In some embodiments, the culture container comprises the microbial growth media of any of the embodiments described herein. In some embodiments, the culture container comprises a sensor for monitoring (e.g., provides a signal proportional to) a parameter of the microbial growth media indicative of the presence or absence of microbial growth in the microbial growth media. In some embodiments, the sensor is a pH sensor. In some embodiments, the sensor is a CO₂ sensor. In some embodiments, the sensor is a dissolved CO₂ sensor. In some embodiments, the sensor is an O₂ sensor. In some embodiments, the sensor is a dye. In some embodiments, the dye is a pH sensitive dye. In some embodiments, the dye is an O₂ sensitive dye. In some embodiments, the sensor is a CO₂, or dissolved CO₂, sensitive dye. In some embodiments, the pH, O₂, CO₂, or dissolved CO₂, sensor is a pH, O₂, CO₂, or dissolved CO₂ sensitive resin. In some embodiments, the culture container has, has about, has at least, has at least about, has not more than, or has not more than about 0.01, 0.025, 0.05, 0.075, 0.1, 0.5, 1.0, 2.0, 3.0, 4.0, 5.0, 6.0, 7.0, 8.0, 9.0 or 10.0 g/L of pH, O₂, CO₂, or dissolved CO₂ sensitive resin, or is a range defined by any two of the preceding values. For example, in some embodiments, the culture container has, or has about, 0.01 to 10.0, 0.01 to 6.5, 0.01 to 4.0, 0.01 to 2.0, 0.01 to 1.0, or 0.01 to 0.10 g/L of pH, O₂, CO₂, or dissolved CO₂ sensitive resin. In some embodiments, the sensor is fluorescent. In some embodiments, the presence of Candida e.g., Candida albicans or Candida glabrata, other yeast and/or other microorganisms in the biological sample causes an increase in the concentration of CO₂ in the headspace gas. In some embodiments, the presence of iron and/or copper in the microbial growth media causes the CO₂ concentration in the headspace gas to increase more rapidly than would occur in culture containers containing microbial growth media without the supplemented iron and/or copper. In some embodiments, the increased CO₂ concentration in the headspace gas alters the pH of the microbial growth media in the culture container. In some embodiments, a sensor monitors (e.g., provides a signal proportional to) the concentration of CO₂. In some embodiments, a sensor monitors (e.g., provides a signal proportional to) the pH of the microbial growth media contained within the culture container. In some embodiments, the pH sensor comprises a pH sensitive dye. In some embodiments, the pH sensor comprises multiple pH sensitive dyes. In some embodiments, the microbial growth media and/or the pH sensor turns a distinct color to indicate a specific pH. In some embodiments, the pH sensor is a fluorescent probe. In some embodiments, the culture container is clear. In some embodiments, the pH of the microbial growth media bottle is monitored by an apparatus. In some embodiments, the pH monitoring instrument comprises a BACTEC FX. In some embodiments, detecting a change in the pH and/or CO₂ of the microbial growth media is indicative of a systemic infection. In some embodiments, the biological sample is further processed to identify the microorganism present in the biological sample. In some embodiments, the subject is administered an appropriate antibiotic regime for the identified microorganism.

In some embodiments, the culture container comprises a gaseous headspace. In some embodiments, the gaseous headspace comprises CO₂, O₂, and N₂. In some embodiments, the gaseous headspace comprises only CO2, O2, and N2 (excluding contaminating gases). In some embodiments, the amount of O₂ in the headspace gas is, is about, is at least, is at least about, is not more than, or is not more than about, 25%, 30%, 40%, 45%, 46%, 47%, 48%, 49%, 50%, 51%, 52%, 53%, 54%, 55%, 56%, 57%, 58%, 59%, 60%, 65%, 70%, 75% O₂, or is a range defined by any two of the preceding values. For example, in some embodiments, the amount of O₂ in the headspace gas is, or is about, 25% to 75%, 47% to 60%, or 52% to 56% O₂. In some embodiments, the amount of CO₂ in the headspace gas is, is about, is at least, is at least about, is not more than, or is not more than about, 15%, 20%, 21%, 22%, 23%, 24%, 24.1%, 24.2%, 24.3%, 24.4%, 24.5%, 24.6%, 24.7%, 24.8%, 24.9%, 25%, 26%, 27%, 28%, 29%, 30%, 40%, 45% CO₂, or is a range defined by any two of the preceding values. For example, in some embodiments, the amount of CO₂ in the headspace gas is, or is about, 15% to 45%, 15% to 30%, 15% to 25%, 20% 25%, or 24% to 25% CO₂. In some embodiments, the amount of N₂ in the headspace gas is the amount remaining after adding an amount of O₂ and CO₂ disclosed herein. In some embodiments, the amount of N₂ in the headspace gas is, is about, is at least, is at least about, is not more than, or is not more than about, 0%, 5%, 10%, 15%, 19%, 20%, 21%, 22%, 23%, 24%, 25%, 26%, 27%, 28%, 28.1%, 28.3%, 28.3%, 28.4%, 28.5%, 28.6%, 28.7%,28.8%, 28.9%, 29%, 30%, 31%, 32%, 33%, 34%, 35%, 40%, 45%, 50%, 55%, or 60% N₂, or is a range defined by any two of the preceding values. For example, in some embodiments, the amount of N₂ in the headspace gas is, or is about, 0% to 60%, 0% to 40%, 0% to 30%, 20% to 40%, or 20% to 30% N₂ In some embodiments, the headspace gas comprises, comprises about, comprises at least, comprises at least about, comprises not more than, or comprises not more than about, 25% to 75% O₂, 15% CO₂ to 45% CO₂, and 0% to 60% N₂, optionally wherein the headspace gas contains only CO₂, O₂, and N₂, (excluding contaminating gases). In some embodiments, the headspace gas comprises, comprises about, comprises at least, comprises at least about, comprises not more than, or comprises not more than about, 52% to 56% O₂, 24% CO₂ to 25% CO₂, and 19% to 24% N₂. In some embodiments, the headspace gas comprises, comprises about, comprises at least, comprises at least about, comprises not more than, or comprises not more than about, 24% CO₂, 47.5% O₂, and 28.5% N₂.

In some embodiments, the addition of iron and/or copper to the microbial growth media as described herein results in increased oxygen consumption by a microorganism grown in the media. In some embodiments, the concentration of oxygen in the headspace gas is chosen for a specific microorganism. In some embodiments, the microorganism is a yeast. In some embodiments, the microorganism is one or more selected from Abiotrophia defectiva, Acinetobacter lwoffii, Agreggatibacter actinomycetemcomitans, Aerococcus viridins, Alcaligenes faecalis, Bacillus subtilis, Candida albicans, Candida glabrata, Cardiobacterium hominis, Corynebacterium jeikeium, Cryptococcus neoformans, Eikinella corrodens, Enterobacter cloacae, Enterococcus faecalis, Escherichia coli, Granulicatella adiacens, Haemophilus influenzae, Haemophilus influenzae, Haemophilus influenzae type a, Haemophilus influenzae type b, Haemophilus parainfluenzae, Kingella kingae, Klebsiella pneumoniae, Leuconostoc species, Micrococcus luteus, Neisseria gonorrhoeae, Neisseria meningitidis, Pediococcus acidilactici, Proteus mirabilis, Providencia stuartii, Pseudomonas aeruginosa, Rothia mucilaginosa, Saccharomyces cerevisiae, Staphylococcus aureus, Staphylococcus epidermidis, Stenotrophomonas maltophilia, Streptococcus agalactiae, Streptococcus pneumoniae, Streptococcus pneumoniae, Streptococcus pneumoniae, Streptococcus pneumoniae, Streptococcus pyogenes, and Streptococcus sanguinis. In some embodiments, the culture container comprises a pH sensor. In some embodiments, the pH sensor is a dye. In some embodiments, the pH sensor is fluorescent. In some embodiments, microbial growth inside the culture container comprising the microbial growth media alters the concentration of CO₂ in the culture container. In some embodiments, microbial growth inside the culture container comprising the microbial growth media increases the concentration of CO₂ in the culture container. In some embodiments, increasing the concentration of CO₂ inside the microbial growth media alters the pH of the enhanced microbial growth media. In some embodiments, the addition of iron and/or copper to the microbial growth media decreases the TTD of Candida e.g., Candida albicans or Candida glabrata, other yeast and/or other microorganisms in a biological sample. In some embodiments, the biological sample is blood. In some embodiments, the biological sample is serum. In some embodiments, the microbial growth media is tested for TTD, percent recovery, DVE, false positive rate, growth support with antimicrobials, or any combination therein.

In some embodiments, a system for detecting the presence or absence of a microbe in a sample is disclosed. In some embodiments, the system comprises a microbial growth culture container as described herein. In some embodiments, the system for detecting the presence or absence of a microbe in a sample comprises a detector for obtaining a signal from the sensor. In some embodiments, system for detecting the presence or absence of a microbe in a sample comprises a computer configured to determine if the signal obtained by the detector indicates that a microbe is present in the microbial growth media. In some embodiments, the system comprises a BACTEC FX instrument.

In some embodiments, a method of culturing a microbial organism is disclosed. In some embodiments, the method comprises inoculating the microbial growth culture medium disclosed herein with a biological sample, and culturing a microbe in the sample in the microbial growth culture medium. In some embodiments, the biological sample is a blood, serum, plasma, or urine sample. In some embodiments the biological sample is obtained from a septic subject. In some embodiments the biological sample is obtained from a systemic inflammatory response syndrome (SIRS)-positive subject. In some embodiments, the inoculating step comprises adding the sample to the microbial growth culture medium in a microbial growth culture container as described herein. In some embodiments, the microbial organism is Candida e.g., Candida albicans or Candida glabrata, other yeast and/or other microorganisms.

In some embodiments, a method of detecting the presence or absence of the microbe in a biological sample is disclosed.. In some embodiments, the biological sample is a blood, serum, plasma, or urine sample. In some embodiments the biological sample is obtained from a septic subject. In some embodiments the biological sample is obtained from a systemic inflammatory response syndrome (SIRS)-positive subject. In some embodiments, a method of detecting the presence or absence of Candida e.g., Candida albicans or Candida glabrata, other yeast and/or other microorganisms in a biological sample is disclosed. In some embodiments, the microorganism is Abiotrophia defectiva, Acinetobacter lwoffii, Agreggatibacter actinomycetemcomitans, Aerococcus viridins, Alcaligenes faecalis, Bacillus subtilis, Candida albicans, Candida glabrata, Cardiobacterium hominis, Corynebacterium jeikeium, Cryptococcus neoformans, Eikinella corrodens, Enterobacter cloacae, Enterococcus faecalis, Escherichia coli, Granulicatella adiacens, Haemophilus influenzae, Haemophilus influenzae, Haemophilus influenzae type a, Haemophilus influenzae type b, Haemophilus parainfluenzae, Kingella kingae, Klebsiella pneumoniae, Leuconostoc species, Micrococcus luteus, Neisseria gonorrhoeae, Neisseria meningitidis, Pediococcus acidilactici, Proteus mirabilis, Providencia stuartii, Pseudomonas aeruginosa, Rothia mucilaginosa, Saccharomyces cerevisiae, Staphylococcus aureus, Staphylococcus epidermidis, Stenotrophomonas maltophilia, Streptococcus agalactiae, Streptococcus pneumoniae, Streptococcus pneumoniae, Streptococcus pneumoniae, Streptococcus pneumoniae, Streptococcus pyogenes, or Streptococcus sanguinis. In some embodiments, the volume of the biological sample is, is about, is at least, is at least about, is not more than, or is not more than about, 0, 0.001, 0.01, 0.1, 0.2, 0.3, 0.4, 0.5, 1, 1.5, 2, 2.5, 3, 3.5, 4, 4.5, 5, 6, 7, 8, 9, or 10 mL, or is a range defined by any two of the preceding values. For example, in some embodiments, the volume of the biological sample is, or is about, 0 to 10 mL, 0 to 5 mL, 0 to 3 mL, 1 to 10 mL, 3 to 10 mL, or 3 to 5 mL. In some embodiments, the apparatus with reduced TTD of Candida comprises a vessel with a body portion. In some embodiments, the body portion of the vessel contains a microbial growth media with reduced TTD ofCandida and a headspace gas. In some embodiments, the microbial growth media comprises DI water, iron, copper, sucrose, dextrose, D-Trehalose, yeast extract, L-glutamic acid, tryptic soy broth, sodium polyanethole sulfonate, menadione, pyridoxal HCl, ferulic acid, sodium hydroxide, ascorbic acid, L-Cysteine, and hemin.

Unless defined otherwise, technical and scientific terms used herein have the same meaning as commonly understood when read in light of the instant disclosure by one of ordinary skill in the art to which the present disclosure belongs. For purposes of the present disclosure, the following terms are explained below.

The articles “a” and “an” are used herein to refer to one or to more than one (for example, at least one) of the grammatical object of the article unless the context indicates otherwise. By way of example, “an element” means one element or more than one element.

By “about” is meant a quantity, level, value, number, frequency, percentage, dimension, size, amount, weight or length that varies by as much as 10% to a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The disclosure is generally disclosed herein using affirmative language to describe the numerous embodiments. The disclosure also includes embodiments in which subject matter is excluded, in full or in part, such as substances or materials, method steps and conditions, protocols, or procedures.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described herein without departing from the scope of the claimed subject matter. All such modifications and changes are intended to fall within the scope of the disclosed subject matter.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.” The use of “A and/or B” is understood to include the possibility of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed herein. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting.

All references cited herein, including but not limited to published and unpublished applications, patents, and literature references, are incorporated herein by reference for the subject matter referenced, and in their entirety and are hereby made a part of this specification. To the extent publications and patents or patent applications incorporated by reference contradict the disclosure contained in the specification, the specification is intended to supersede and/or take precedence over any such contradictory material.

### EXAMPLES

Some aspects of the embodiments discussed herein are disclosed in further detail in the following examples, which are not in any way intended to limit the scope of the present disclosure. Those in the art will appreciate that many other embodiments also fall within the scope of the invention, as it is described herein and in the claims.

### Example 1

### Media Formulation and Bottling

In one representative example, a culture medium was formulated to decrease the time to detection (TTD) of yeast and other microorganisms in BD Plus Aerobic/26F BACTEC bottles. The culture media comprised 20L DI water, 2 g ferric ammonium citrate, 0.02 g cupric sulfate, 6.6 g sucrose, 40 g dextrose, 40 g D-trchalose, 50 g yeast extract, 10 g L-glutamic acid, 550 g TSB, 10 g SPS, 0.01 g menadione, 0.2 g pyridoxal HCl, 0.1 g ferulic acid, 15.31 g sodium hydroxide, 1 g ascorbic acid, 0.01 g L-cysteine, and 0.1 g hemin. The media further comprised 0.1875 g sodium hydroxide and 20 ml DI water for the hemin solution; 0.2 g sodium hydroxide and 20 mL water for the ferulic acid solution; and, 0.78 mL ethanol for the menadione solution. The hemin, menadione, and ferulic acid solutions were all prepared no more than 72 hours prior to preparation of the culture media and stored at between about 2-8 C. The hemin solution was prepared by pipetting 20 ml of DI water into a 50 mL Falcon tube. The sodium hydroxide was added to the tube and vortexed. Once the sodium hydroxide was dissolved, the hemin was added to the tube and vortexed. Once the hemin was dissolved, the solution was labeled with an expiration time of 72 hours and stored in the refrigerator at between about 2-8 C. The menadione solution was prepared by pipetting 780 μL of ethanol into a conical tube, adding the amount of previously weighed menadione to the tube, capping the tube, and vortexing until dissolved. Once the menadione was dissolved, the tube was labeled with an expiration time of 72 hours and stored in the refrigerator at 2-8 C. The ferulic acid solution was prepared by pipetting 20 mL DI water into a 50 mL Falcon tube, adding the sodium hydroxide, and vortexing until the sodium hydroxide was dissolved. Once dissolved, the ferulic acid was added to the tube and the solution was vortex until the ferulic acid was fully dissolved. Once dissolved, the tube was labeled with a 72 hour expiration and stored in the refrigerator at between about 2-8 C.

In this example, bead baths were preheated to approximately 70 C. and a pH meter was turned on and calibrated. Bottles for containing the formulated media were prewarmed in the water bath. 12 L of DI water, a magnetic stir bar, and a thermometer were added to a 45 L carboy container situated on top of a magnetic hot plate. The mixture was stirred at between about 200 and 300 rpm and brought to a temperature of between 45 C. and 75 C. The solution was held between 45 C. and 75 C. for the duration of the formulation procedure. Sucrose, dextrose, d-trehalose, yeast extract, and L-glutamic acid were each then sequentially added to the pot. 3 L of DI water was added to the container in order to rinse down any of the added components remaining on the container walls. TSB was then slowly, in order to minimize dust production, added to the 45 L container, followed by an additional 3 L of DI water. The TSB was allowed to fully dissolve for more than 10 minutes before sequentially adding the SPS, menadione solution, pyridoxal HCl, and the ferulic acid solution to the container. 1 L of DI water was used to rinse down the sides of the container before adding the sodium hydroxide, ascorbic acid, L-cysteine, ferric ammonium citrate, and cupric sulfate to the container. The temperature of the container was then raised to 75 C.+/−3 C. The hemin solution was pipetted into the container. 1 L of DI water was used to rinse down the container, and the solution was stirred until the hemin solution was fully dissolved.

In this example, 40-50 mL of the formulated media was removed from the container for pH testing and allowed to cool to 55 C. Once cooled, the pH of the media was tested and adjusted using NaOH or HCl to raise or lower the pH such that the pH of the cooled media was between 7.70 and 7.80, which translates to a room temperature pH of 7.1-7.4 after adding a Plus Aerobic gas mixture to the headspace upon bottling, autoclaving, and storing for between about 1 and 7 days. The Plus Aerobic gas mixture comprised 24% CO₂, 47.5% O₂, and 28.5% N₂. To bottle the media, a media dispenser was activated and allowed to run until media was continuously being dispensed at between 67 C. and 78 C. The pre-warmed media fill bottles were removed from the bead bath one at a time, the bottles being strategically added and removed to the bead bath to ensure that each bottle has a similar time to pre-warm. The formulated media and the Plus Aerobic Gas mixture at between about 5-10 psi were dispensed into the pre-warmed fill bottles using a 3.5″ F-nozzle and a 5 second hold time. A rubber stopper was placed in the bottle to indicate how far the nozzle can go into a bottle without disturbing the resin. The nozzle mouth should reach just far enough to pass the bottle neck. It is critical to have space in between the bottle mouth and nozzle so that gas can be blown out. Each bottle received a 1.75 g CO₂ sensor, 4.6 g of resin, and 30 mL media. The filled bottle was then capped and sealed with an automated crimper. Filled bottles were then sterilized using an air over pressure autoclave. Filled, sterilized bottles were stored at room temperature.

### Time to Detection (TTD) Testing

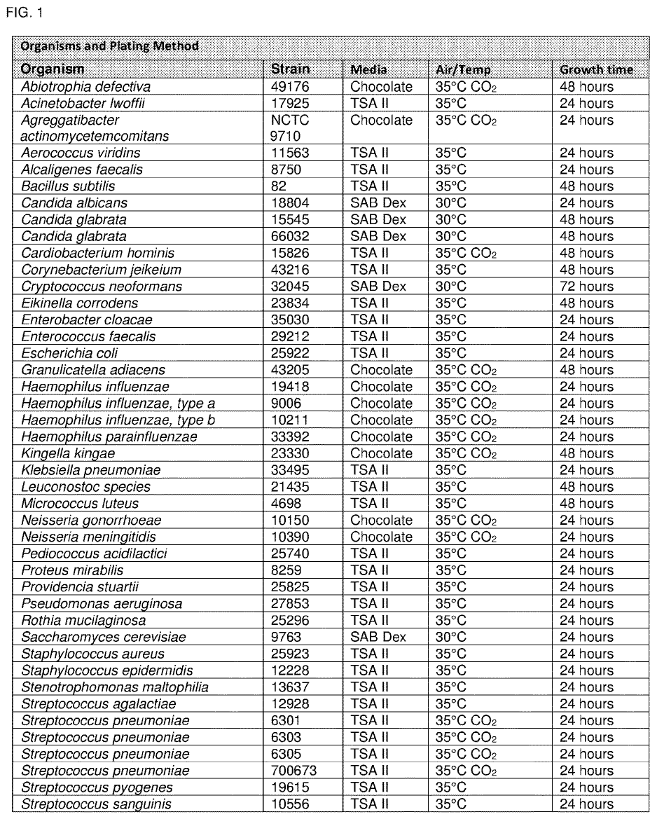

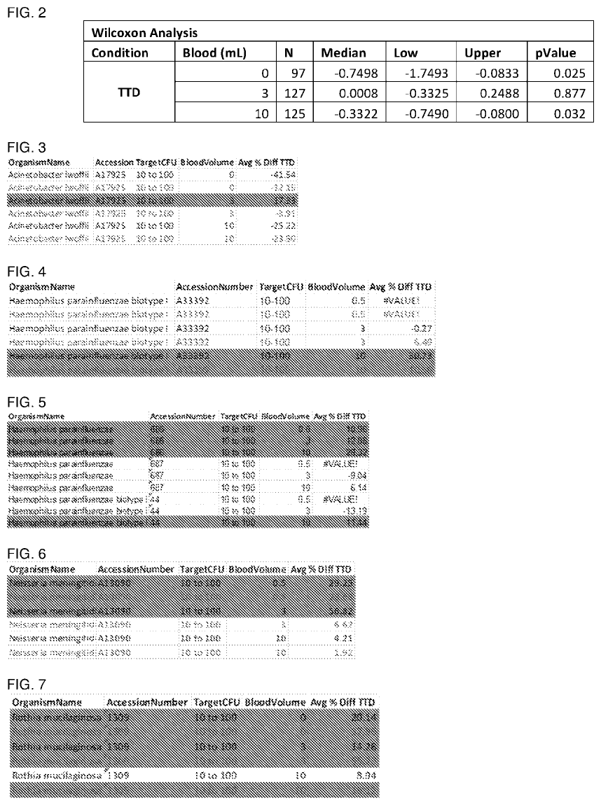

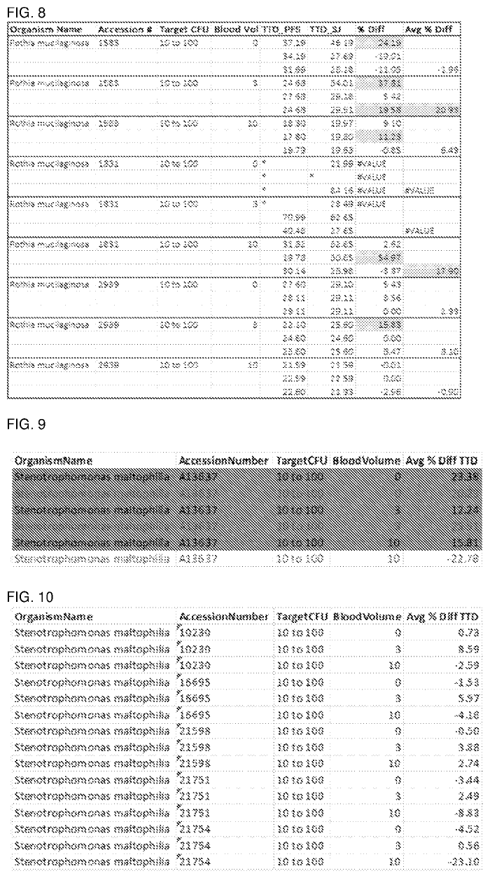

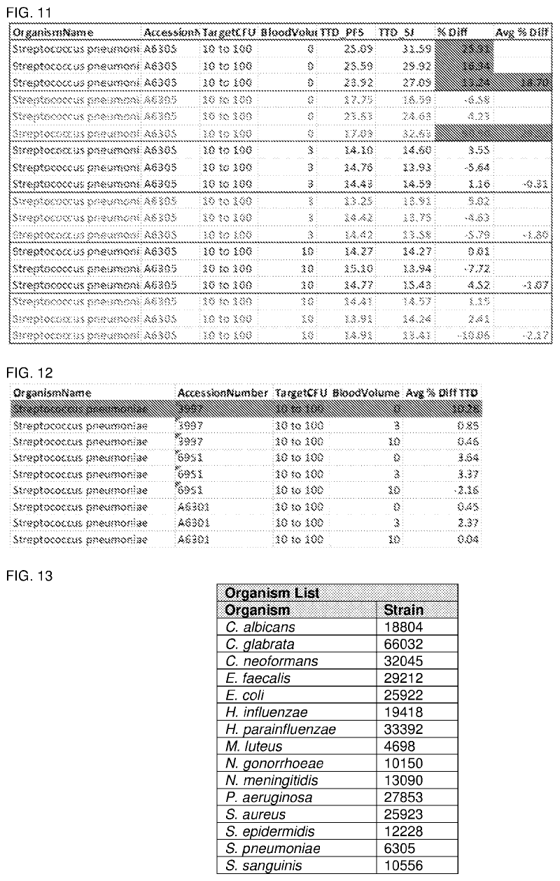

Testing of the formulated culture media was performed using Plus Aerobic/26F BACTEC bottles that had been manufactured within a week of the media formulation as a control. Biological testing comprised time to detection (TTD), percent recovery (sensitivity), false positive rate, false negative rate, delayed vial entry (DVE), and growth support with antimicrobials. TTD testing was performed using 43 organisms with a target CFU of 10-100 in blood volumes of 0 or 0.5 mL, organism dependent, 3 mL, and 10 mL. The testing was run in replicates of three. Prior to the day of testing, the bottles were racked and labeled, and the organisms were sub-cultured accordingly. FIG. 1 provides a list of the organisms tested, the strain, media, and growth conditions. Each organism to be tested was grown overnight on an appropriate plated medium. Each organism was carefully checked for purity. If there was a contaminant or the colonies had an unusual appearance, a Gram stain and/or identification was performed to confirm the identity. The organism is re-cultured. 0.1 mL of the final organism dilution was dispensed and spread on the appropriate agar for plate counts. Using aseptic technique, each bottle containing 30 mL of media was amended with 0.5, 3, or 10 mL of bagged human blood using the appropriate syringe and needle. Paired sets were inoculated with blood from the same donor unit at the same time. The bottles were then inoculated with 0.1 mL of the appropriate organism. After inoculation, the bottles were loaded onto a BACTEC FX instrument using a standard protocol length of 120 hours. Paired bottles were entered into the FX at the same time in adjacent positions. The specification for TTD testing was no relevant difference from the Plus Aerobic/26F BACTEC media as determined by Wilcoxon analysis. TTD was assessed by Wilcoxon paired analysis and the comparison based on the median of the interval. Paired bottles were included only when both bottles detected growth within the 120 hour protocol. The median TTDs had to either have no statistically detectable difference, i.e., a p-value of less than 0.05, or in test conditions where a statistically relevant detectable difference occurred, they should not favor the Plus Aerobic/26F BACTEC media. Relevant difference is defined as no greater than 10% difference in time to detection when in favor of Plus Aerobic/26F BACTEC media. Pass criterion is based on collective performance using all the organisms, but individual organisms that fall outside the 10% difference limit are addressed. The results of this experiment are listed in FIG. 2. The results for each biological test had results that were acceptable per the criteria outlined in each specification. There were several organisms that did not meet the 10% difference criterion. The individual organisms that did not pass the acceptance criteria were: A. lwoffii, H. parainfluenzae, N. meningitidis, R. mucilaginosa, S. maltophilia, and S. pneumoniae.The organisms were retested along with additional strains in some instances. A. lwoffii had a significantly longer TTD in favor of the control in 3 mL of blood. The other two blood volumes had an average % difference in TTD that favored the new media formulation. The results of this experiment are listed in FIG. 3. The organism was repeated as an inoculation error was suspected. On repeat (results are in light grey), the results for 3 mL favored the newly formulated media bottles. H. parainfluenzae results showed a significantly longer TTD in the bottles containing 10 mL of blood. The results of this experiment are illustrated in FIG. 4. The organism was repeated in all 3 blood volumes. The repeat results (light grey) for 10 mL of blood still favored the current media. Haemophilus was expected to demonstrate longer TTDs in the bottles containing the newly formulated media due to the increased O₂ and its sensitivity to high levels of oxygen. Three additional strains of H. parainfluenzae were tested. The results of these experiments are listed in FIG. 5. One of the strains showed a preference for Plus Aerobic/26F BACTEC media in all 3 blood volumes. The second strain showed equivalent performance between Plus Aerobic/26F BACTEC media and the new media formulation and the third strain results were split between the 2 media types. In addition, as the oxygen levels decrease, the performance in the bottles with the formulated media was anticipated to improve. N. meningitidis had a >10% difference in TTD in favor of Plus Aerobic/26F BACTEC media for 0.5 mL and 3 mL of blood. This organism was repeated. The repeat results still did not meet the pass criteria for 0.5 mL, but was acceptable at 3 mL. The results of this experiment are listed in FIG. 6. This organism's TTDs are also expected to improve as the oxygen levels in decrease in the headspace gas. R. mucilaginosa's initial results showed a significantly longer TTD in bottles without blood and in bottles with 3 mL. The organism was repeated. Upon retest, the bottles containing the new media formulation had significantly longer TTD's for all blood volumes. The results of these experiments are illustrated in FIG. 7. Three additional strains of R. mucilaginosa were tested. The results of this experiment are listed in FIG. 8. The results highlighted in grey did not pass the 10% acceptance criteria. Some of the results appear to be outliers due to contamination or an inoculation error. The new media formulation exhibited better recovery in one strain. The average of all the strains is within 10% of the Plus Aerobic/26F BACTEC. S. maltophilia had significantly longer TTDs in all the blood volumes in the initial testing. The results of these experiments are illustrated in FIG. 9. The repeat results still showed a significantly longer TTD in the bottles containing no blood and 3 mL of blood. The results were concerning as S. maltophilia carries known resistance to many antimicrobial therapies. Five additional strains were selected for testing. The results of these experiments are listed in FIG. 10. The average TTD for each strain and blood volume tested was below the 10% criteria. The ATCC strain used for testing reacts differently to the newly formulated media than the other strains tested and may not be representative of clinical strains and their TTD in the new media formulation. S. pneumoniae results showed a significantly longer TTD at 0 mL. The organism was repeated using all 3 blood volumes. The results of these experiments are listed in FIG. 11. On repeat, the results were acceptable with the exception of one replicate at 0 mL. The result is most likely due to an inoculation error. Three additional strains of S. pneumoniae were tested as part of the organism list. The results of these experiments are listed in FIG. 12. The data from this experiment demonstrates that the new media formulation has equivalent to enhanced performance when comparing TTDs to Plus Aerobic/26F BACTEC media which contains not more than about 0.2 ppm iron and not more than about 0.025 ppm copper.

### Percent Recovery

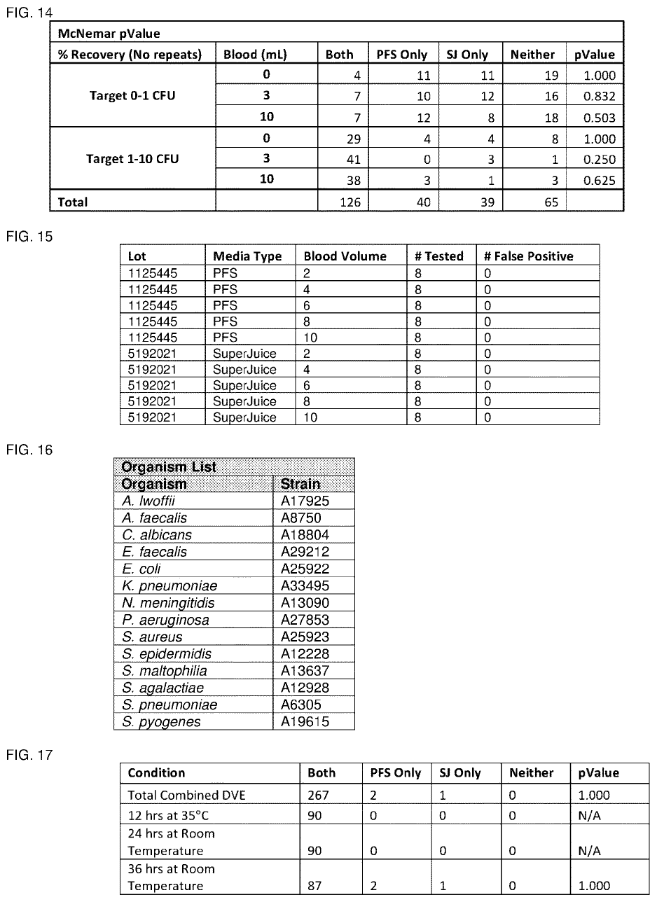

Recovery testing was performed using 15 organisms with target CFUs of 0-1 and 1-10 in blood volumes of 0 mL or 0.5 mL (organism dependent), 3 mL and 10 mL. The testing was run in replicates of 3. The organisms tested in these experiments are listed in FIG. 13. Prior to the day of testing, the bottles were racked and labeled, and the organisms were sub-cultured accordingly. Each organism to be tested was grown overnight on an appropriate plated medium. Each organism was carefully checked for purity. If there was a contaminant or the colonies had an unusual appearance, a Gram stain and/or identification was performed to confirm the identity. 0.1 mL of the final organism dilution was dispensed and spread on the appropriate agar for plate counts. Using aseptic technique, each bottle containing 30 ml of microbial growth media was amended with 0.5, 3 or 10 mL of bagged human blood using the appropriate syringe and needle. Paired sets were inoculated with blood from the same donor unit at the same time. The bottles were inoculated with 0.1 mL of the appropriate organism. After inoculation, the bottles were loaded onto the BACTEC FX instrument using the standard protocol length of 120 hours. Paired bottles were entered into the FX at the same time in adjacent positions. The specification for percent recovery was no relevant difference from current media. Recovery was assessed by McNemar's Chi Square test at 95% confidence level that there was no statistical difference (P<0.5). Any statistical difference that favors the control, would be determined to be relevant which would require determination of clinical relevance. The results of the MeNemar's Chi Square analysis are listed in FIG. 14. Each pValue is >0.05 indicating no significant difference in results. The results for % Recovery were accepted.

### False Positive Rate

False positive rate determination was performed by inoculating the media with either 2, 4, 6, 8, or 10 mL of freshly drawn, aseptic blood. Each blood volume was tested in replicates of 8 using newly formulated media and Plus Aerobic/ 26F BACTEC bottles containing 30 ml of microbial growth media. Each paired set received blood from the same donor at the same time. After blood addition, the bottles were loaded onto a BACTEC FX instrument using a standard protocol length of 120 hours. Paired bottles were entered into the FX at the same time in adjacent positions. The specification was a false positive rate equivalent to Plus Aerobic/26F BACTEC media. The potential for an increase in false positive results due to differences in signal output was evaluated by producing expected negative bottles with different levels of fresh blood and demonstrating that there was no difference in the observed rate of false positive determinations (Chi Square analysis). The results of the Chi Square analysis are listed in FIG. 15. None of the bottles inoculated with fresh blood had a positive result therefore a p-value could not be calculated.

### Delayed Vial Entry (DVE)

Three DVE conditions that were tested are as follows: 1) Inoculated bottles incubated for 12 hours at 35±1° C; 2) inoculated bottles incubated on the lab bench for 24 hours at room temperature (25±2.5° C.); and, 3) inoculated bottles incubated on the lab bench for 36 hours at room temperature (25±2.5° C.). Each of the DVE conditions were tested with 14 organisms with a target CFU of 10-100 in blood volumes of 3 mL and 10 mL. The testing was run in replicates of 3. The organisms used in DVE testing are listed FIG. 16. Prior to the day of testing, the bottles were racked and labeled, and the organisms were sub-cultured accordingly. Each organism to be tested was grown overnight on an appropriate plated medium. Each organism was carefully checked for purity. If there was a contaminant or the colonies had an unusual appearance, a Gram stain and/or identification was performed to confirm the identity.0.1 mL of the final organism dilution was dispensed and spread on the appropriate agar for plate counts. Using aseptic technique, each bottle containing 30 ml of microbial growth media was amended with 3 or 10 mL of bagged human blood using the appropriate syringe and needle. Paired sets were inoculated with blood from the same donor unit at the same time. The bottles were inoculated with 0.1 mL of the appropriate organism. After inoculation, the bottles were loaded onto a BACTEC FX instrument using a standard protocol length of 120 hours. Paired bottles were entered into the FX at the same time in adjacent positions at the same time. The specification was equivalent to Plus Aerobic/26F BACTEC media or no relevant difference from Plus Aerobic/26F BACTEC. DVE Recovery was assessed by McNemar's Chi Square test at a 95% confidence level that there was no difference (p≤0.05). Relevant is defined as a statistically lower DVE recovery in the newly formulated media compared to the reference media using a pValue of <0.05. The results of the McNemar's Chi Square analysis are listed in FIG. 17. The pValue for the 36 hour at room temperature condition was 1.000 indicating there is not significant difference. A pValue could not be calculated for 12 hours at 35° C. and 24 hours at room temperature because every bottle had a positive result. The DVE results passed the verification and validation criteria.

### Growth Support With Antimicrobials

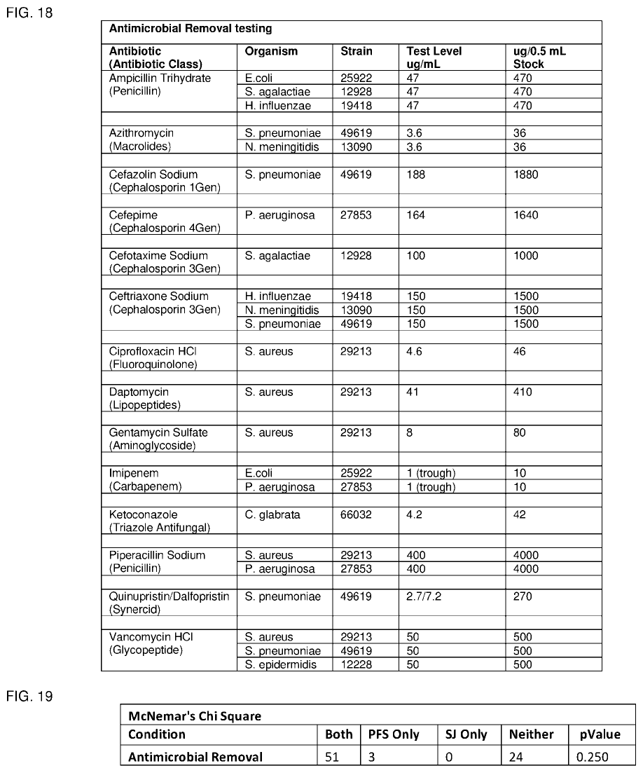

Two conditions were tested with nine organisms. The test had a target CFU of 10-100. 14 antibiotics were used. A blood volume of 10 mL was used. The testing was run in replicates of three. FIG. 18 lists the organisms and antibiotics used along with the test levels. Prior to the day of testing, the bottles were racked and labeled, and the organisms were sub-cultured accordingly. The master and working stocks of the antibiotics were prepared and stored appropriately. Each organism to be tested was grown overnight on an appropriate plated medium. Each organism was carefully checked for purity. If there was a contaminant or the colonies had an unusual appearance, a Gram stain and/or identification was performed to confirm the identity. 0.1 mL of the final organism dilution was dispensed and spread on the appropriate agar for plate counts. Using aseptic technique, each bottle containing 30 ml of microbial growth media was amended with 3 or 10 ml of bagged human blood using the appropriate syringe and needle. Paired sets were inoculated with blood from the same donor unit at the same time. The bottles were inoculated with 0.1 mL of the appropriate organism and 0.5 of the appropriate antibiotic working solution. After inoculation, the bottles were loaded onto a BACTEC FX instrument using a standard protocol length of 120 hours. Paired bottles were entered into the FX at the same time in adjacent positions. The specification was equivalent antimicrobial removal in the newly formulated media as compared to Plus Aerobic/26F BACTEC. No relevant difference from current media was defined by one of the following test result conditions: 1) the number of microbes detected in newly formulated medium is greater than the number detected in the Plus Aerobic/26F BACTEC media; or 2) McNemar's Chi Square test results in a p value >0.05 if test condition 1 is not met. The results of the McNemar's Chi Square analysis are listed in FIG. 19. Condition 1 was not met. One out of 6 bottles was positive for E. coli and Imipenem. The one bottle was the Plus Aerobic/26F BACTEC control. Two out of 6 bottles turned positive for S. pneumoniae and Vancomycin. The bottles were the Plus Aerobic/26F BACTEC control. Vancomycin and Imipenem absorption is poor with our resins and is a known test risk. As condition 1 was not met, the pValue was calculated. The pValue was 0.250 which is greater than 0.05 indicating the difference is not significant. The antimicrobial growth support testing passed the verification and validation criterion. The data from this Example clearly demonstrated that the new media formulation has enhanced performance when comparing TTDs to the Plus Aerobic/26F BACTEC. Further, the new media formulation has equivalent to enhanced false positive rate, percent recovery, DVE, and growth support with microbials as compared to Plus Aerobic/26F BACTEC.

### Example 2

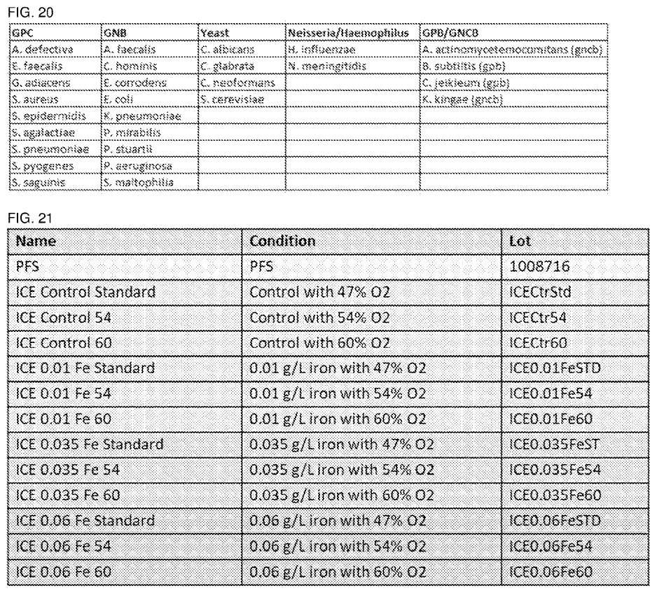

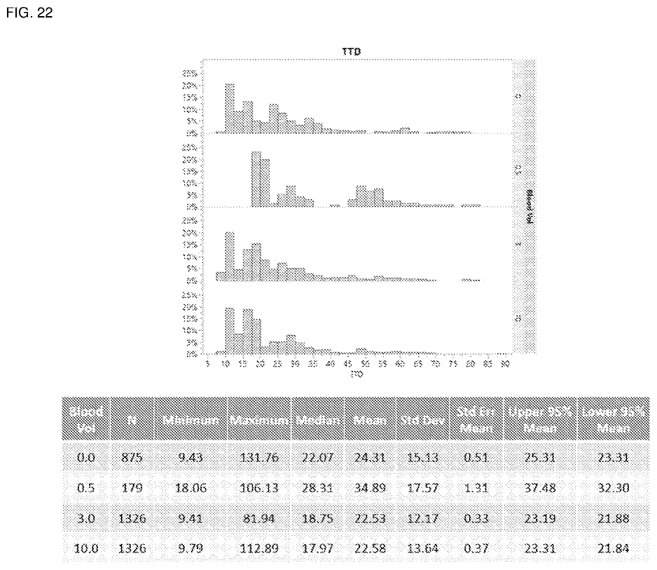

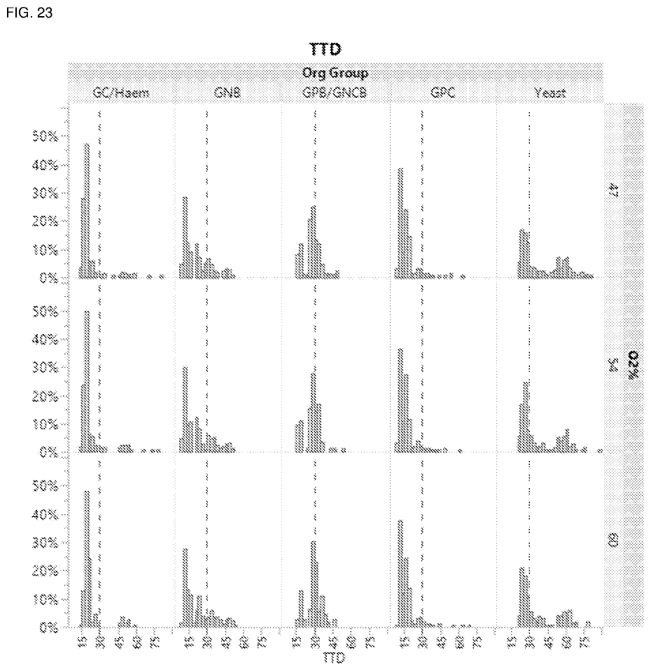

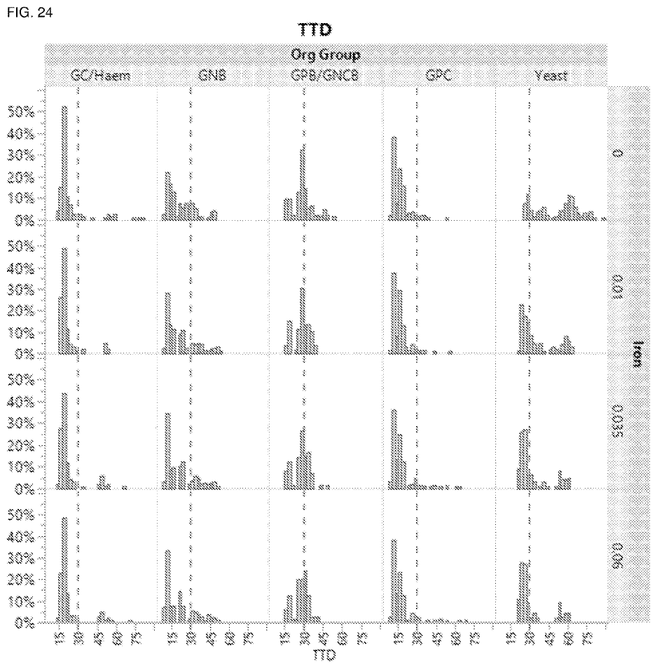

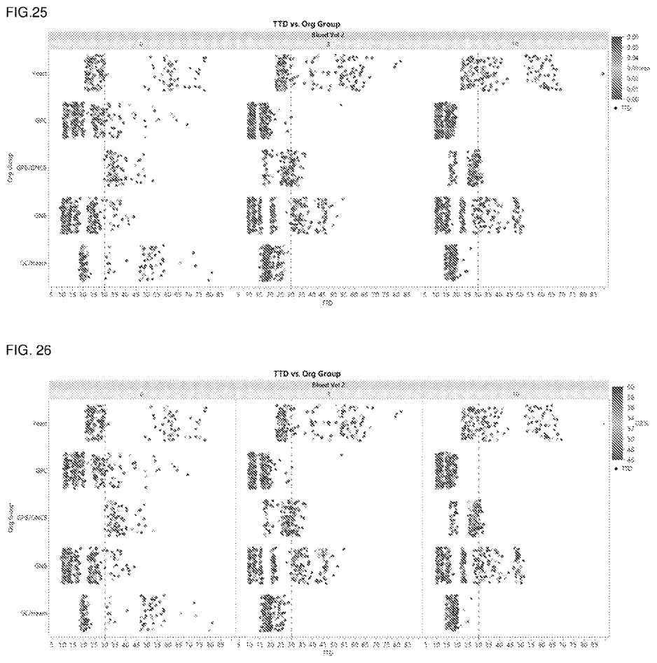

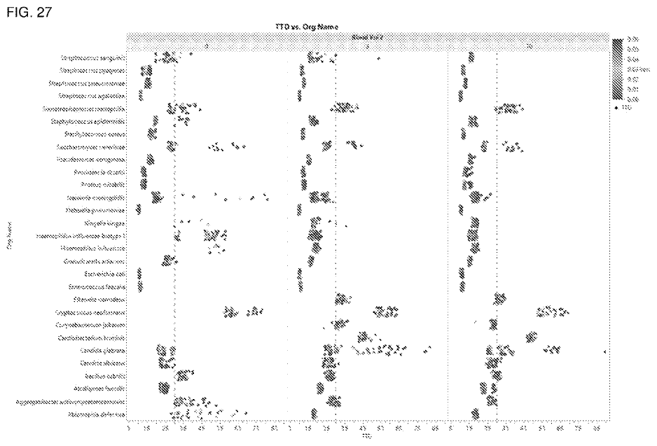

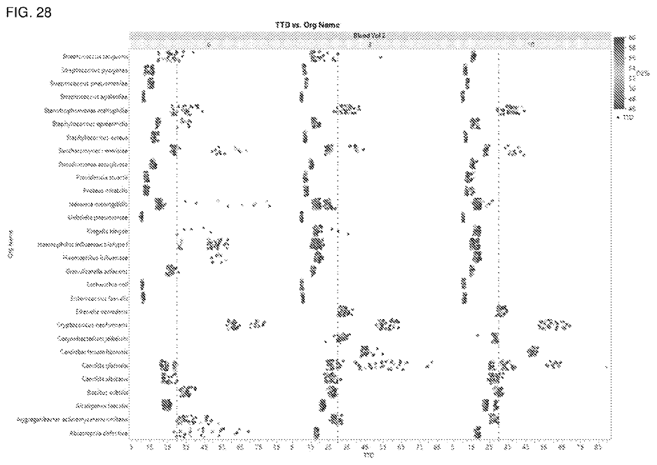

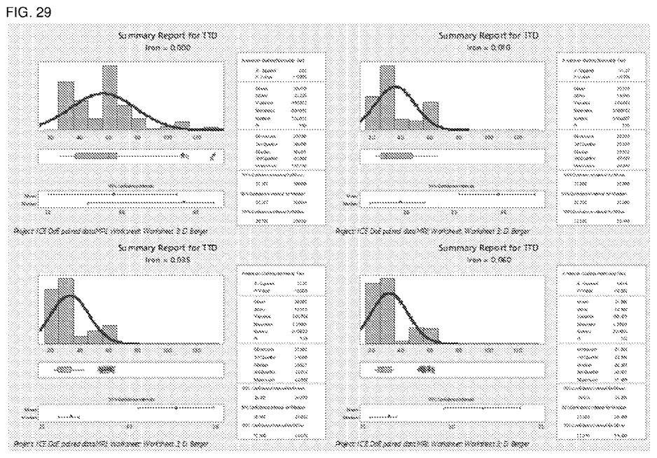

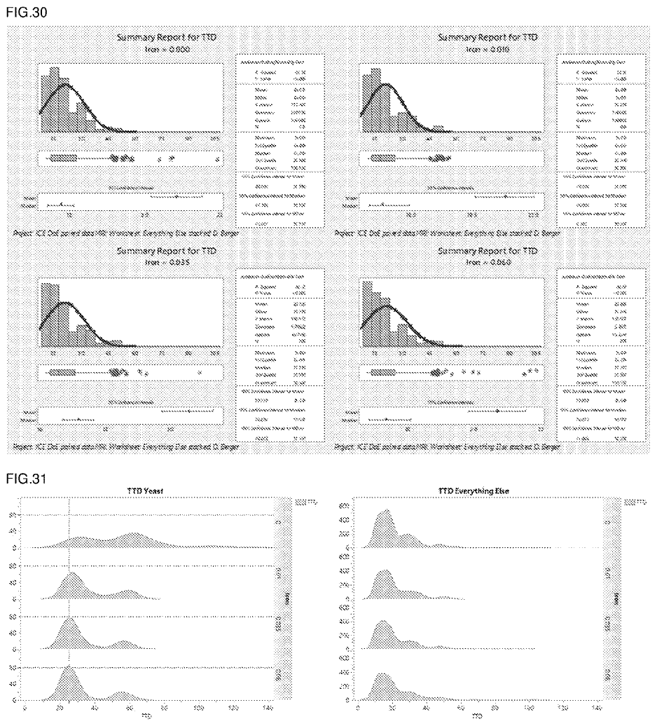

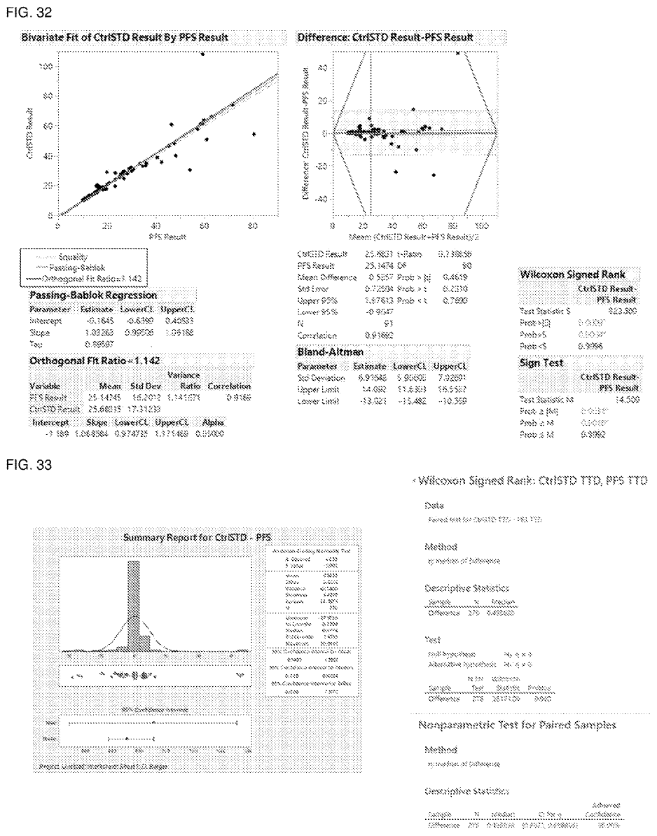

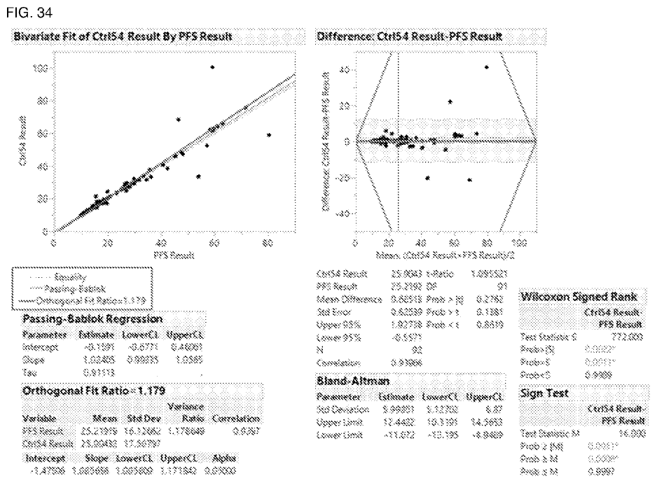

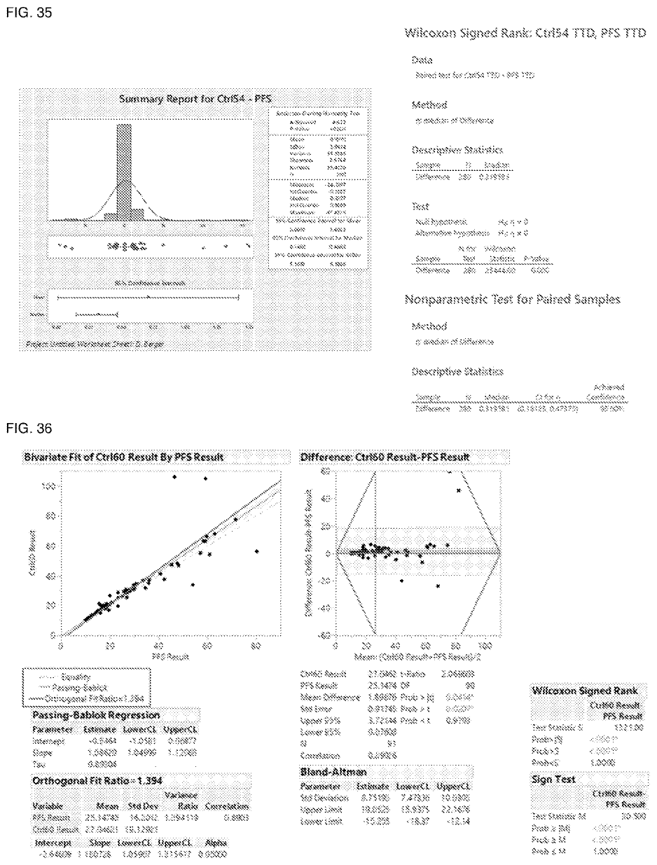

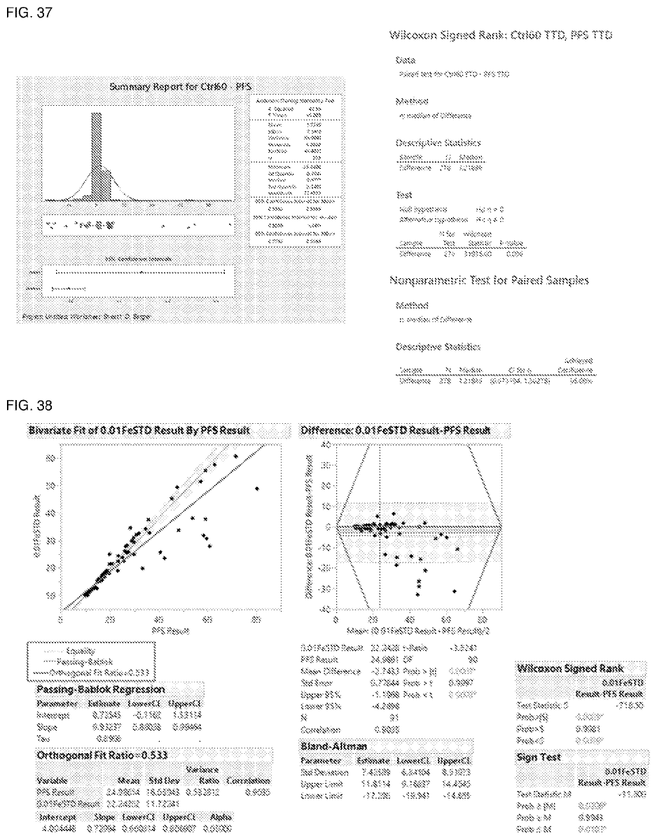

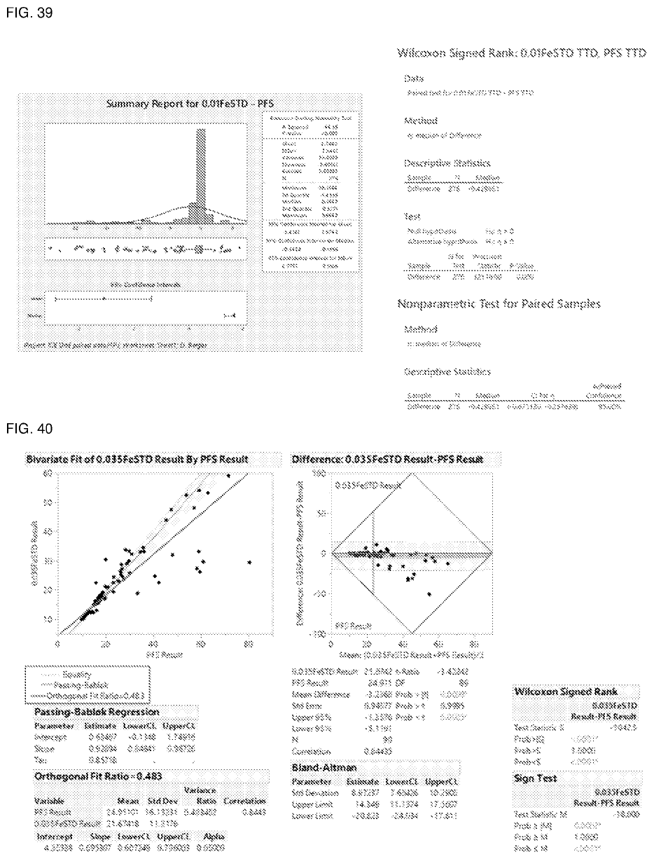

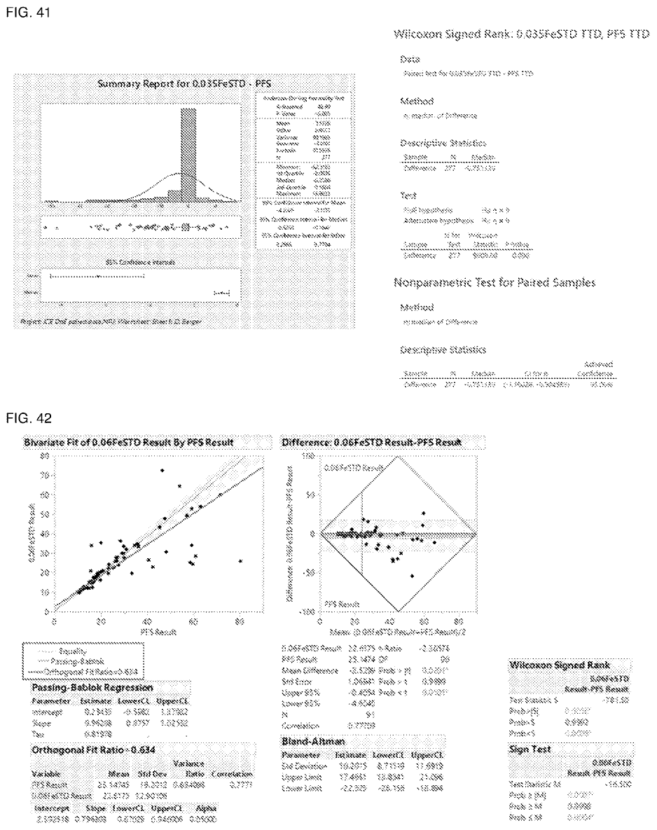

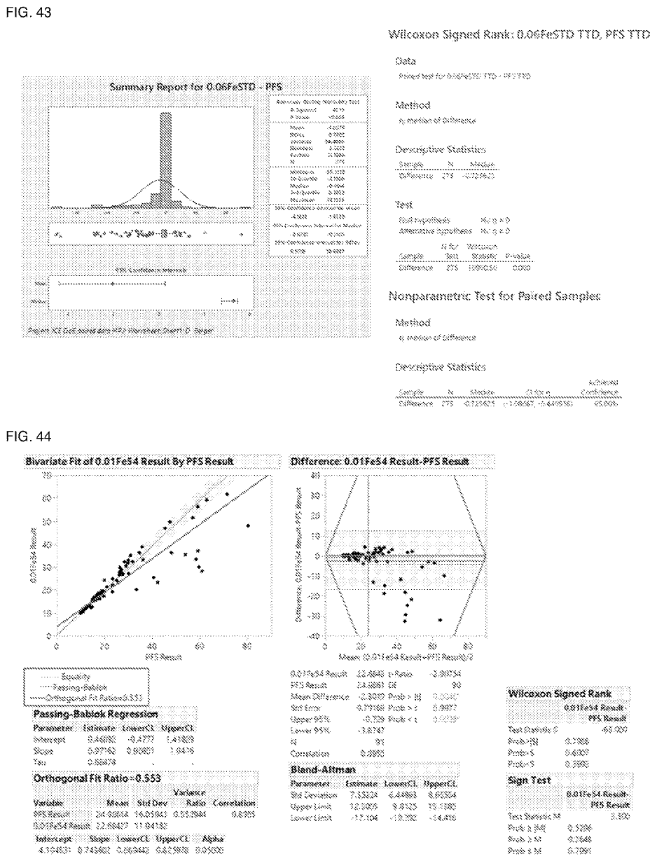

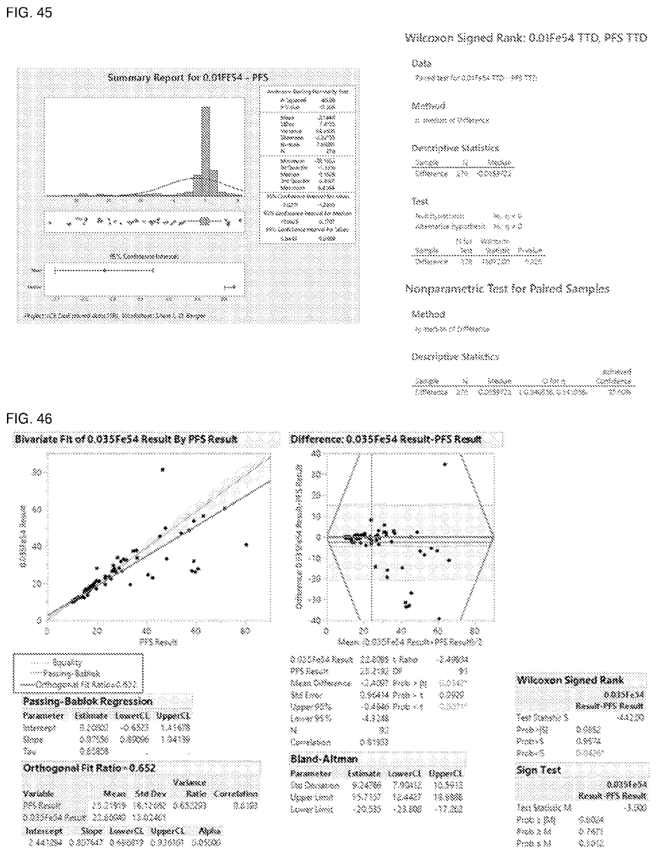

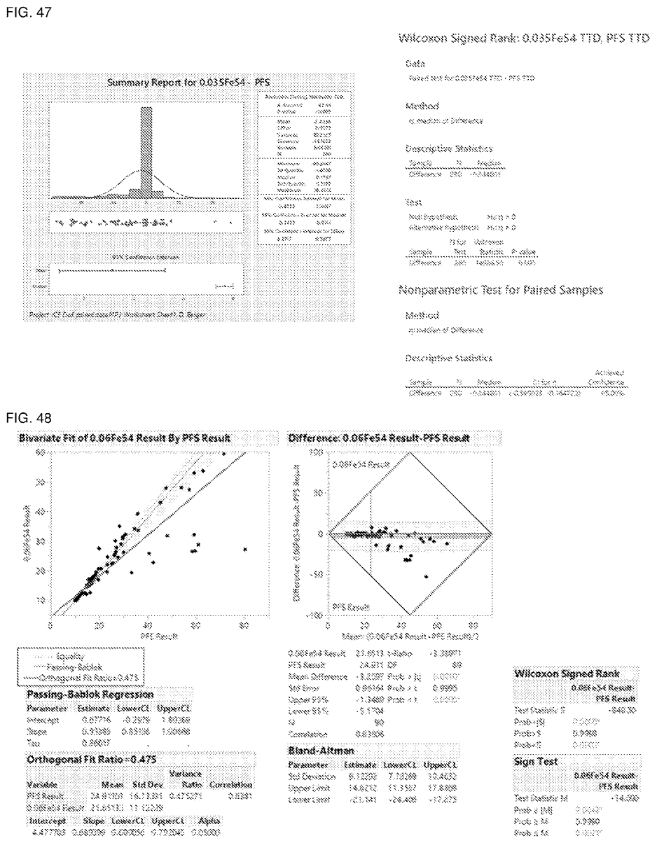

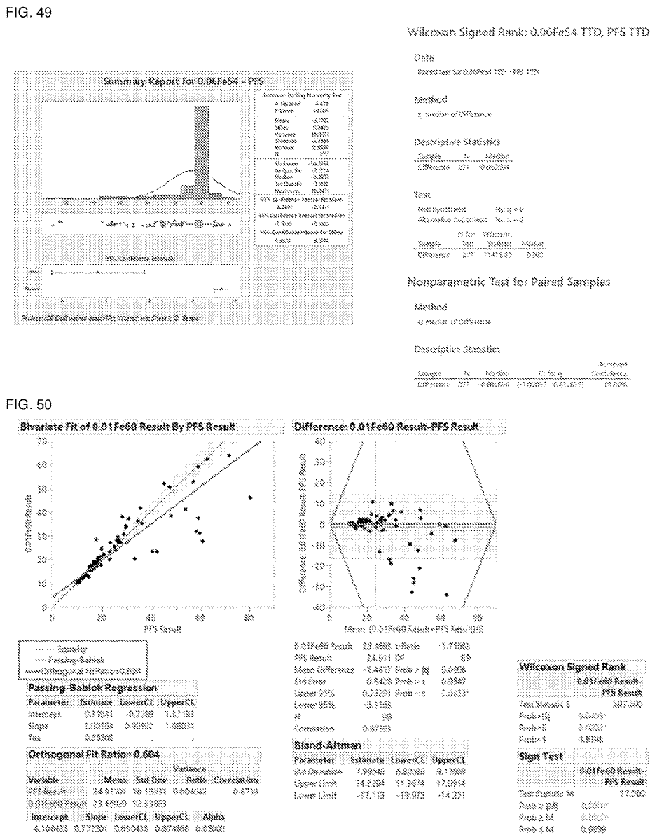

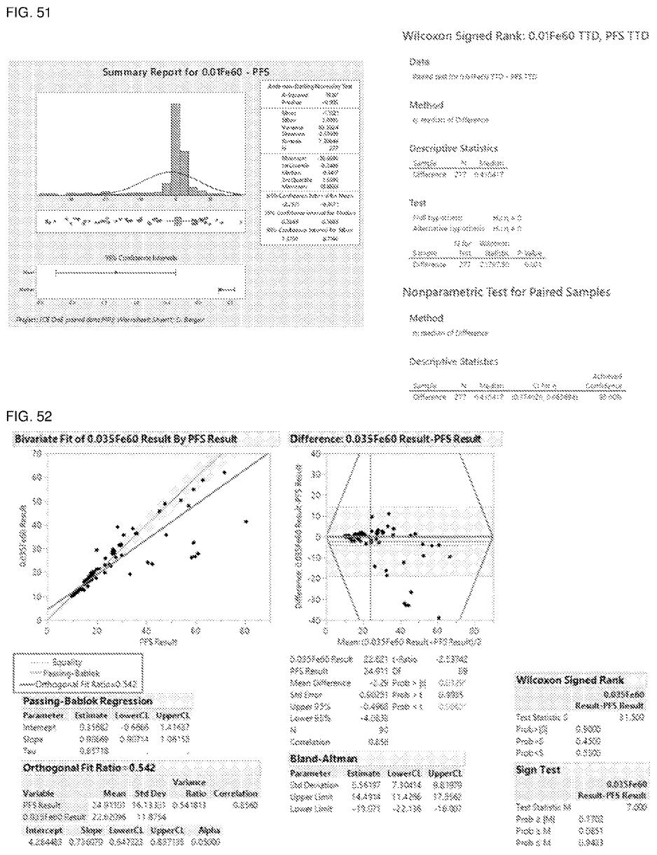

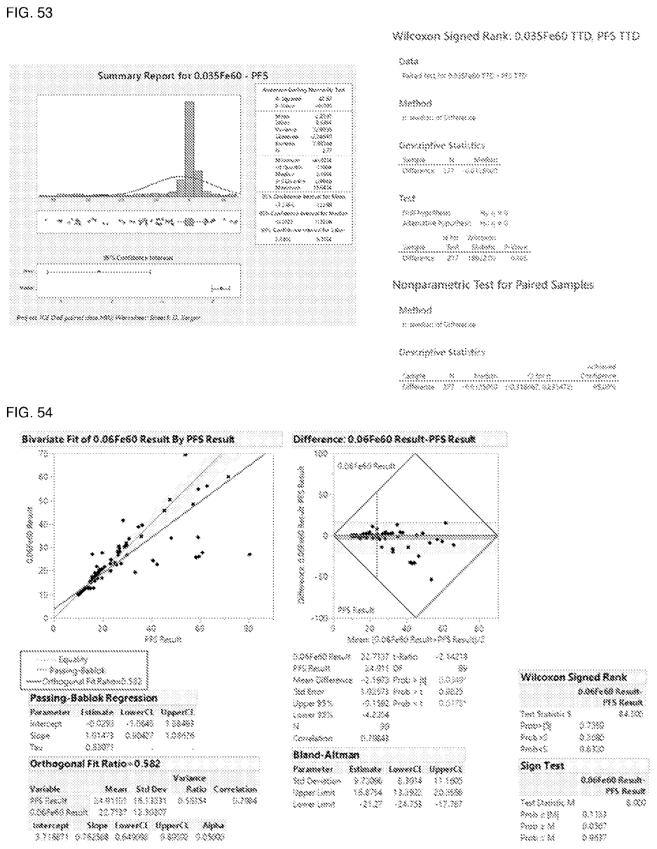

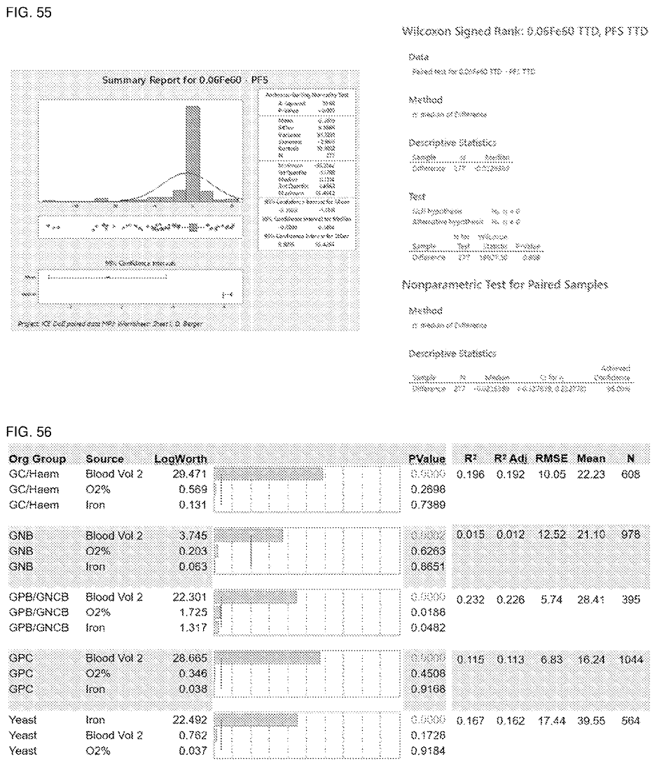

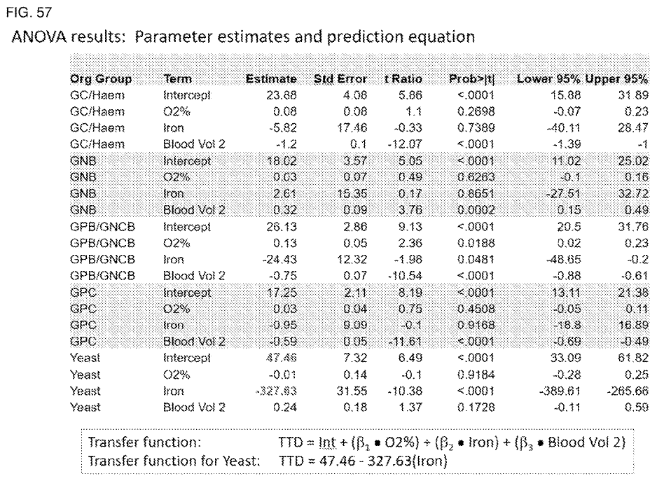

In one representative example of the present disclosure, the ratio of iron to oxygen was optimized to promote improved TTD of yeast in the culture media while maintaining a shelf life equivalent to Plus Aerobic/26F BACTEC. TTD testing was performed using 28 organisms with a target CFU of 10-100 in blood volumes of 0 or 0.5 mL, organism dependent, 3 mL, and 10 mL. The organisms tested are illustrated in FIG. 20. Four different concentrations of iron provided as ferric ammonium citrate (0 g/L, 0.01 g/L, 0.035 g/L, and 0.06 g/L) in the media otherwise as described in Example 1, and three different concentrations of oxygen (47%, 54%, and 60%) were tested. FIG. 21 illustrates the combination of conditions tested. The testing was run in replicates of three. Prior to the day of testing, the bottles were racked and labeled, and the organisms were sub-cultured accordingly. Each organism to be tested was grown overnight on an appropriate plated medium. Each organism was carefully checked for purity. If there was a contaminant or the colonies had an unusual appearance, a Gram stain and/or identification was performed to confirm the identity. The organism is then re-cultured. 0.1 mL of the final organism dilution was dispensed and spread on the appropriate agar for plate counts. Using aseptic technique, each bottle containing 30 ml of microbial growth media was amended with 0, 0.5, 3, or 10 mL of bagged human blood using the appropriate syringe and needle. Paired sets were inoculated with blood from the same donor unit at the same time. The bottles were then inoculated with 0.1 mL of the appropriate organism. After inoculation, the bottles were loaded onto a BACTEC FX instrument using a standard protocol length of 120 hours. Paired bottles were entered into the FX at the same time in adjacent positions. FIG. 22 shows histograms illustrating the TTD of microorganisms in blood volumes of 0, 0.5, 3, and 10 mL. Although there is some variability in TTD with different blood volumes, the mean TTD across all blood volumes and organisms was approximately 24-27 hours. FIG. 23 shows histograms illustrating the TTD of 5 different classes of microorganisms (GC/Haem or Neisseria/Haemophilius, GNB or Gram-negative bacilli, GPB/GNCB or Gram-positive bacilli/Gram-negative coccobacilli, GPC or Gram-positive cocci, and yeast) at three concentrations of O 2(47%, 54%, and 60%) in the gaseous headspace. The dotted vertical line in each panel at a TTD of 30 hours represents the approximate TTD of all organisms. FIG. 24 shows histograms illustrating the TTD of GC/Haem, GNB, GPB/GNCB, GPC, and yeast at each of four different concentrations of iron in the newly formulated media (0 g/L, 0.01 g/L, 0.035 g/L, and 0.06 g/L). The dotted vertical line in each panel at a TTD of 30 hours represents the approximate TTD of all organisms. FIG. 25 shows dot plots illustrating the TTD of GC/Haem, GNB, GPB/GNCB, GPC, and yeast microorganisms in blood volumes of 0/0.5, 3, and 10 mL and microbial growth media comprising at 0, 0.01, 0.035, and 0.06 g/L iron. The dotted vertical line in each panel at a TTD of 30 hours represents the approximate TTD of all organisms. Increasing iron concentration in the media from 0 to 0.06 g/L decreased the TTD of yeast and other microorganisms, with an optimal iron concentration of about 0.035 g/L (light gray dots). FIG. 26 shows dot plots illustrating the TTD of GC/Haem, GNB, GPB/GNCB, GPC, and yeast microorganisms in blood volumes of 0/0.5, 3, and 10 mL and microbial growth media bottle headspace gas comprising 47%, 54%, or 60% O₂. The dotted vertical line in each panel at a TTD of 30 hours represents the approximate TTD of all organisms. Increasing oxygen concentration in the microbial growth media bottle headspace gas from 47% to 60% O₂ decreased the TTD of yeast and other microorganisms, with an optimal gaseous headspace O₂ concentration of about 54% (light gray dots). FIG. 27 shows dot plots illustrating the TTD of each of the 28 organisms tested in blood volumes of 0/0.5, 3, and 10 mL and microbial growth media comprising at 0, 0.01, 0.035, and 0.06 g/L iron. The dotted vertical line in each panel at a TTD of 30 hours represents the approximate TTD of all organisms. Increasing iron concentration in the microbial growth media from 0 to 0.06 g/L decreased the TTD of Candida glabrata, Candida albicans, and other microorganisms, with an optimal iron concentration of about 0.035 g/L (light gray dots). FIG. 28 shows dot plots illustrating the TTD of each of the 28 organisms tested in blood volumes of 0/0.5, 3, and 10 mL and microbial growth media bottle headspace gas comprising 47%, 54%, or 60% O₂. The dotted vertical line in each panel at a TTD of 30 hours represents the approximate TTD of all organisms. Increasing oxygen concentration in the microbial growth media bottle headspace gas from 47% to 60% O₂ decreased the TTD Candida glabrata, Candida albicans, and other microorganisms, with an optimal gaseous headspace O₂ concentration of about 54% (light gray dots). FIG. 29 shows summary histograms for the TTD of yeast in microbial growth media comprising 0, 0.01, 0.035, and 0.06 g/L iron. FIG. 30 shows summary histograms for the TTD of all other microorganisms tested in microbial growth media comprising 0, 0.01, 0.035, and 0.06 g/L iron. FIG. 31 shows histograms illustrating the improved TTD of yeast 2901 as compared to the TTD of all other microorganisms 2902 in microbial growth media comprising 0, 0.01, 0.035, and 0.06 g/L iron. Increasing iron concentration in the media from 0 to 0.06 g/L decreased the TTD of yeast and other microorganisms, with an optimal iron concentration of about 0.035 g/L. FIGS. 33-55 show bivariate fits and summary histograms of the TTD of microorganisms in the Plus Aerobic/26F BACTEC; newly formulated media in microbial growth media bottles with 47%, 54%, and 60% O₂ in the gaseous headspace; and newly formulated microbial growth media comprising 0, 0.01, 0.035, and 0.06 g/L iron in microbial growth media bottles with 47%, 54%, and 60% O₂ in the gaseous headspace. The data from each experiment was organized for statistical testing via a general linear model analysis of variance (ANOVA). Blood volumes of 0 or 0.5, 3, and 10 mL were recoded as 0, 3, and 10 mL to help balance the design. Additional models were assessed holding the blood volume constant. The effects and parameters of the ANOVA arc shown in FIGS. 56 and 57. FIG. 56 illustrates the results of the ANOVA analyzing the effects of the tested conditions on TTD. FIG. 57 illustrates the results of the ANOVA parameter estimates and prediction equation. There was an expectation that only the yeast category would show a significant effect with the iron addition. Other categories were monitored to ensure no adverse effects. The results indicate that iron lowers the TTD of yeasts and may have benefits with other organisms as well.

What is claimed is:

1. A microbial growth media providing an increased growth rate of Candida, the microbial growth media comprising:
a concentration of iron of about 1 to about 400 μM; and
optionally, a concentration of copper of up to about 700 μM;
wherein the growth rate of Candida cultured in the microbial growth media is increased as compared to the same microbial growth media in the absence of the iron and copper.

2. The microbial growth media of claim 1, wherein the time to detection (TTD) of Candida cultured in the microbial growth media is reduced as compared to the same microbial growth media in the absence of the iron and copper.

3. The microbial growth media of any one of the preceding claims, wherein the reduced TTD of Candida cultured in the microbial growth media is determined by:
amending culture containers comprising the microbial growth media with and without the iron and copper with 0.5, 3, or 10 ml of blood;
inoculating the culture containers with 0.1 mL of a culture of Candida to provide 10-100 CFU of Candida per container;
culturing the Candida at about 30° C. to 37° C. for up to 120 hours;
monitoring the culture containers for a signal indicative of the presence of Candida; and
determining the TTD of the Candida in the microbial growth media with and without the iron and copper.

4. The microbial growth media of any one of the preceding claims, wherein the Candida is Candida albicans or Candida glabrata.

5. The microbial growth media of any one of the preceding claims, wherein the microbial growth media comprises, or comprises about, 0, 0.01, 0.1, 0.2, 0.3, 0.4, 0.5, 0.6, 0.7, 0.8, 0.9, or 1.0 g/L yeast nitrogen base, or a range defined by any two of the preceding values.

6. The microbial growth media of any one of the preceding claims, wherein the microbial growth media comprises less than 0.01% w/v yeast nitrogen base.

7. The microbial growth media of any one of the preceding claims, wherein the yeast nitrogen base does not comprise histidine, methionine, and tryptophan.

8. The microbial growth media of any one of the preceding claims, wherein the microbial growth media comprises, or comprises about, 1, 2, 3, 4, 5, 6, 7, 8, 9, 10, 15, 20, 25, 28, 50, 75, 100, 125, 150, 170, 175, 200, 225, 250, 275, 300, 325, 350, or 400 μM iron, or a range defined by any two of the preceding values, optionally 1 to 400 μM, 1 to 350 μM, 1 to 200 μM, 1 to 100 μM, 1 to 50 μM, 2 to 400 μM, 2 to 350 μM, 25 to 400 μM, 25 to 325 μM, 25 to 200μM, 25 to 100 μM, or 25 to 75 μM.

9. The microbial growth media of any one of the preceding claims, wherein concentration of iron is about 28 μM.

10. The microbial growth media of any one of the preceding claims, wherein concentration of iron is about 100 μM.

11. The microbial growth media of any one of the preceding claims, wherein concentration of iron is about 170 μM.

12. The microbial growth media of any one of the preceding claims, wherein the iron is ferric iron.

13. The microbial growth media of any one of the preceding claims, wherein the iron is provided as ferric ammonium citrate, ferric chloride, ferric sulfate, ferric nitrate.

14. The microbial growth media of any one of the preceding claims, wherein the microbial growth media comprises, or comprises about, 0, 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 75, 100, 150, 200, 300, 400, 500, 600, 650, or 700 μM copper, or a range defined by any two of the preceding values, optionally 0 to 700 μM, 0 to 650 μM, 0 to 500 μM, 0 to 300 μM, 0 to 100 μM, 0 to 50 μM, 0 to 25 μM, 0.1 to 100 μM, 0.1 to 50 μM, 0.1 to 25 μM, 0.5 to 650 μM, 0.5 to 300 μM, 0.5 to 100 μM, 0.5 to 50 μM, or 0.5 to 25 μM.

15. The microbial growth media of any one of the preceding claims, wherein the concentration of copper is about 3 μM.

16. The microbial growth media of any one of the preceding claims, wherein the copper is cupric copper.

17. The microbial growth media of any one of the preceding claims, wherein the copper is provided as cupric sulfate, cupric chloride, cupric nitrate, cupric bromide, cupric chlorate, and/or copper(II) gluconate.

18. The microbial growth media of any one of the preceding claims, wherein the microbial growth media is an aqueous liquid growth media further comprising sucrose, dextrose, D-Trehalose, yeast extract, L-glutamic acid, tryptic soy broth, sodium polyanethole sulfonate, menadione, pyridoxal HCl, ferulic acid, sodium hydroxide, ascorbic acid, L-Cysteine, hemin.

19. The microbial growth media of any one of the preceding claims, wherein the microbial growth medium comprises, or comprises about, 1.0 to 10.0 g/L yeast extract, 0.15 to 1.5 g/L sucrose, 1.0 to 10.0 g/L dextrose, 1.0 to 10.0 g/L D-trehalose, 0.001 to 1.5 g/L L-glutamic acid, 0.00001 to 0.01 g/L L-cysteine, 0.5 to 15 g/L TSB, 0.001 to 1.5 g/L SPS, 0.00001 to 0.01 g/L menadione, 0.0001 to 1 g/L Pyridoxal HCl, 0.0001 to 1 g/L ferulic acid, 0.1 to 10 g/L sodium hydroxide, 0.001 to 1.0 g/L ascorbic acid, and 0.0001 to 1 g/L hemin, in water.

20. The microbial growth media of any one of the preceding claims, wherein the microbial growth medium comprises, or comprises about, 0.001 g/L to 0.15 g/L iron, 0.0001 to 0.001 g/L copper, 3.0 to 5.0 g/L yeast extract, 0.15 to 1.5 g/L sucrose, 2.5 to 3.5 g/L dextrose,

1. 5 to 3.5 g/L D-trehalose, 0.5 to 1 g/L L-glutamic acid, 0.0001 to 0.0012 g/L L-cysteine, 0.5 to 60 g/L TSB, 0.5 to 1.5 g/L SPS, 0.0004 to 0.0012 g/L menadione, 0.005 to 0.025 g/L Pyridoxal HCl, 0.001 to 0.1 g/L ferulic acid, 0.5 to 3 g/L sodium hydroxide, 0.01 to 0.25 g/L ascorbic acid, and 0.001 to 0.1 g/L hemin, in water.

21. The microbial growth media of any one of the preceding claims, wherein the microbial growth media comprises, or comprises about, 0.01 g/L iron; 0.001 g/L copper; 0.528 g/L sucrose; 3.2 g/L dextrose; 3.2 g/L D-trehalose; 4 g/L yeast extract; 0.8 g/L L-glutamic acid; 44 g/L TSB; 0.8 g/L SPS, 0.0008 g/L menadione, 0.016 g/L Pyridoxal HCl; 0.008 g/L ferulic acid; 1.225 g/L sodium hydroxide; 0.08 g/L ascorbic acid; 0.0008 g/L L-cysteine, and 0.008 g/L g hemin.

22. The microbial growth media of any one of the preceding claims, wherein the microbial growth media comprises, or comprises about, 0.01 g/L iron; 0.001 g/L copper; 0.33 g/L sucrose; 2 g/L dextrose; 2 g/L D-trehalose; 2.5 g/L yeast extract; 0.5 g/L L-glutamic acid;

27. 5 g/L TSB; 0.5 g/L SPS, 0.00005 g/L menadione, 0.01 g/L Pyridoxal HCl; 0.005 g/L ferulic acid; 0.7655 g/L sodium hydroxide; 0.05 g/L ascorbic acid; 0.0005 g/L L-cysteine, and 0.005 g/L hemin.

23. The microbial growth media of any one of the preceding claims, wherein the microbial growth media is able to support the growth of one or more microbial species selected from the group consisting of Abiotrophia defectiva, Acinetobacter lwoffii, Agreggatibacter actinomycetemcomitans, Aerococcus viridins, Alcaligenes faecalis, Bacillus subtilis, Candida albicans, Candida glabrata, Cardiobacterium hominis, Corynebacterium jeikeium, Cryptococcus neoformans, Eikinella corrodens, Enterobacter cloacae, Enterococcus faecalis, Escherichia coli, Granulicatella adiacens, Haemophilus influenzae, Haemophilus influenzae, Haemophilus influenzae type a, Haemophilus influenzae type b, Haemophilus parainfluenzae, Kingella kingae, Klebsiella pneumonia, Leuconostoc species, Micrococcus luteus, Neisseria gonorrhoeae, Neisseria meningitidis, Pediococcus acidilactici, Proteus mirabilis, Providencia stuartii, Pseudomonas aeruginosa, Rothia mucilaginosa, Saccharomyces cerevisiae, Staphylococcus aureus, Staphylococcus epidermidis, Stenotrophomonas maltophilia, Streptococcus agalactiae, Streptococcus pneumoniae, Streptococcus pneumoniae, Streptococcus pneumoniae, Streptococcus pneumoniae, Streptococcus pyogenes, and Streptococcus sanguinis.

24. The microbial growth media of any one of the preceding claims, wherein the increase in growth rate of Candida is, is about, is at least, is at least about, 10%, 15%, 20%, 25%, 30%, 35%, 40%, 45%, 50%, 60%, 70%, 75%, or 80%,, or is a range defined by any two of the preceding values.

25. The microbial growth media of any one of the preceding claims, wherein the reduction in TTD of Candida is, is about, is at least, is at least about, 10%, 15%, 20%, 25%, 30%, 35%, 40%, 45%, 50%, 60%, 70%, 75%, or 80%, or is a range defined by any two of the preceding values.

26. The microbial growth media of any one of the preceding claims, wherein the reduction in TTD of Candida is, is about, is at least, is at least about, 1, 2, 3, 4, 5, 6, 7, 8, 9, 10,12, 15, 18, 24, 30, 36, 48, 60, or 72 hours, or is a range defined by any two of the preceding values.

27. A culture container for detecting growth of a microbe, the container comprising:
the microbial growth media of any one of the preceding claims; and
a sensor for monitoring a parameter of the microbial growth media indicative of microbial growth in the microbial growth media.

28. The culture container of claim 27, wherein the sensor is separated from the contents of the microbial growth media by a permeable membrane.

29. The culture container of claim 27 or 28, wherein the parameter monitored is pH, O₂, and/or CO₂.

30. The culture container of any one of claims 27 to 29, wherein the sensor comprises a pH sensor.

31. The culture container of claim 30, wherein the pH sensor comprises a fluorescent, phosphorescent, or colorimetric pH responsive agent.

32. The culture container of any one of claims 27 to 31, wherein the sensor comprises a O₂ sensor.

33. The culture container of claim 32, wherein the pH sensor comprises a fluorescent, phosphorescent, or colorimetric O₂ responsive agent.

34. The culture container of any one of claims 27 to 33, wherein the sensor comprises a CO₂ sensor.

35. The culture container of claim 34, wherein the CO₂ sensor comprises a fluorescent, phosphorescent, or colorimetric pH responsive agent.

36. The culture container of any one of claims 27 to 35, wherein the sensor comprises a pH, O₂, and/or CO₂ sensitive resin.

37. The culture container of claim 36, wherein the culture container comprises an amount of pH and/or CO₂ sensitive resin that is, is about, is at least, is at least about, is not more than, or is not more than about, 0.01, 0.025, 0.05, 0.075, 0.1, 0.5, 1.0, 2.0, 3.0, 4.0, 5.0, 6.0, 7.0, 8.0, 9.0 or 10.0 g/L, or a range defined by any two of the preceding values.

38. The culture container of any one of claims 27 to 37, wherein the container further comprises a headspace volume comprising a gaseous mixture of O₂, CO₂, and N₂.

39. The culture container of claim 38, wherein the headspace gas comprises:
about 25% to about 75% O₂,
about 15% to about 45% CO₂, and
about 0% to about 40% N₂.

40. The culture container of claim 38 or 39, wherein the headspace gas comprises, comprises about, comprises at least, comprises at least about, comprises less than, or comprises less than about 25%, 30%, 40%, 45%, 46%, 47%, 48%, 49%, 50%, 51%, 52%, 53%, 54%, 55%, 56%, 57%, 58%, 59%, 60%, 65%, 70%, 75% O₂, or a range defined by any two of the preceding values.

41. The culture container of claim 38 or 39, wherein the headspace gas comprises about 47% to 60% O₂.

42. The culture container of claim 38 or 39, wherein the headspace gas comprises about 52% to 56% O₂.

43. The culture container of any one of claims 38 to 42, wherein the headspace gas comprises, comprises about, comprises at least, comprises at least about, comprises less than, or comprises less than about 15%, 20%, 21%, 22%, 23%, 24%, 24.1%, 24.2%, 24.3%, 24.4%, 24.5%, 24.6%, 24.7%, 24.8%, 24.9%, 25%, 26%, 27%, 28%, 29%, 30%, 40%, 45% CO₂, or a range defined by any two of the preceding values.

44. The culture container any one of claims 38 to 42, wherein the headspace gas comprises about 20% to about 30% CO₂.

45. The culture container any one of claims 38 to 42, wherein the headspace gas comprises about 24% to about 25% CO₂.

46. The culture container of any one of claims 36 to 45, wherein the headspace gas comprises, comprises about, comprises at least, comprises at least about, comprises less than, or comprises less than about 20%, 25%, 26%, 27%, 28%, 28.1%, 28.3%, 28.3%, 28.4%, 28.5%, 28.6%, 28.7%,28.8%, 28.9%, 29%, 30%, 31%, 32%, 33%, 34%, 35%, or 40% N₂, or a range defined by any two of the preceding values.

47. The culture container of any one of claims 38 to 45, wherein the headspace gas comprises about 0% to 30% N₂.

48. The culture container of any one of claims 38 to 45, wherein the headspace gas consists of, or consists essentially of, O₂, CO₂, and N₂.

49. The culture container of any one of claims 27 to 48, wherein the microbial growth is growth of a microbe selected from the group consisting of Abiotrophia defectiva, Acinetobacter lwoffii, Agreggatibacter actinomycetemcomitans, Aerococcus viridins, Alcaligenes faecalis, Bacillus subtilis, Candida albicans, Candida glabrata, Cardiobacterium hominis, Corynebacterium jeikeium, Cryptococcus neoformans, Eikinella corrodens, Enterobacter cloacae, Enterococcus faecalis, Escherichia coli, Granulicatella adiacens, Haemophilus influenzae, Haemophilus influenzae, Haemophilus influenzae type a, Haemophilus influenzae type b, Haemophilus parainfluenzae, Kingella kingae, Klebsiella pneumoniae, Leuconostoc species, Micrococcus luteus, Neisseria gonorrhoeae, Neisseria meningitidis, Pediococcus acidilactici, Proteus mirabilis, Providencia stuartii, Pseudomonas aeruginosa, Rothia mucilaginosa, Saccharomyces cerevisiae, Staphylococcus aureus, Staphylococcus epidermidis, Stenotrophomonas maltophilia, Streptococcus agalactiae, Streptococcus pneumoniae, Streptococcus pneumoniae, Streptococcus pneumoniae, Streptococcus pneumoniae, Streptococcus pyogenes, and Streptococcus sanguinis.

50. A system for detecting the presence or absence of a microbe in a sample, the system comprising:
the culture container of any one of claims 27 to 49;
a detector for obtaining a signal from the sensor;
a computer configured to determine if the signal obtained by the detector indicates that a microbe is present in the microbial growth media.

51. A method of culturing a microbe in a sample, the method comprising:
inoculating the microbial growth culture medium of any one of the preceding claims with a sample, and culturing a microbe in the sample in the microbial growth culture medium.

52. The method of claim 51, wherein the method further comprises detecting the presence or absence of a microbe in the sample.

53. The method of claim 51 or 52, wherein the inoculating step comprises adding the sample to the microbial growth culture medium in the culture container of any one of claims 27 to 50.

54. The method of claim 53, wherein detecting the presence or absence of the microbe in the sample comprises monitoring the sensor for a signal indicative of microbial growth in the microbial growth media.

55. The method of claim 54, wherein the signal indicates a change in pH of the microbial growth media.

56. The method of claim 54, wherein the signal indicates a change in the CO₂ of the culture container headspace gas.

57. The method of any one of claims 52 to 55, wherein the microbe is Candida, and the TTD of Candida is reduced as compared to culturing Candida in the same microbial growth media wherein the amount of iron is less than about 0.4 ppm and the amount of copper is less than about 0.04 ppm, or wherein iron and copper are absent from the media.

58. The method of any one of claims 51 to 56, wherein said culturing comprises maintaining the microbial culture medium at a temperature of 35° C. to 39° C., or 37° C.

59. The method of any one of claims 51 to 58, wherein the method is performed using the system of claim 50.

60. The method of any one of claims 51 to 59, wherein the sample is selected from the group consisting of, a biological sample, for example, blood, serum, plasma, urine, cerebrospinal fluid, pleural fluid, chest fluid, thoracentesis fluid, peritoneal fluid, abdominal fluid, ascites, pericardial fluid, bone marrow, synovial fluid, or an industrial sample, for example food or pharmaceutical ingredients.

61. The culture container of any one of claims 51 to 60, wherein the sample is a biological sample from a systemic inflammatory response syndrome (SIRS)-positive or septic patient.
